(19) United States
(12) Patent Application Publication
(10) Pub. No.: US 2023/0027822 A1
(43) Pub. Date: Jan. 26, 2023

(54) Method for Constructing Antibody Complementarity Determining Region Library

(71) Applicant: Nanjing GenScript Biotech Co., Ltd., Nanjing, Jiangsu (CN)

(72) Inventors: Xia Sheng, Nanjing, Jiangsu (CN); Cheng-Hsien Wu, Nanjing, Jiangsu (CN); Yikai Qiu, Nanjing, Jiangsu (CN)

(21) Appl. No.: 17/757,110

(22) PCT Filed: Dec. 13, 2020
(86) PCT No.: PCT/CN2020/135992

§ 371 Date: Jun. 9, 2022

(30) Foreign Application Priority Data
Dec. 13, 2019 (CN) 201911281696.9

(51) Int. Cl. C12N 15/10; C07K 16/22; C12N 15/70; G16B 20/30

(52) U.S. Cl. CPC C12N 15/1089; C07K 16/22; C12N 15/70; C12N 15/1068; G16B 20/30

(57) ABSTRACT

Disclosed are a method and a device for constructing an antibody complementarity determining region (CDR) library. Also disclosed are a method, a device and a computer program product for determining the occurrence frequency of member sequences of an antibody CDR library, by means of which an antibody CDR library with a specific amino acid distribution at one or more positions can be obtained.

### TECHNICAL FIELD

The present invention relate to a method and a device for constructing an antibody complementarity determining region (CDR) library. The present invention also relate to a method, a device and a computer program product for determining the occurrence frequency of member sequences of an antibody CDR library, by means of which an antibody CDR library with a specific amino acid distribution at one or more positions can be obtained.

### BACKGROUND ART

The technique that can reproducibly generate a target-specific antibody is an important innovation for biomedical research and disease diagnosis medicine. Hybridoma technique (Kohler G and Milstein C (1975) Nature 256, 495-497), as an efficient and mature technique for generating a mouse monoclonal antibody, has still been used for generating antibodies for a variety of uses, including therapeutic antibodies. In recent years, some methods have been developed for generating a target-specific antibody in vitro. Among them, the development of in vitro display technique (Bradbury AR et al. (2011) Nat. Biotechnol. 29, 245-254), such as phage display, is of greatest concern, which makes the rapid isolation of a target-specific antibody from a large antibody library become possible. In vitro display method has advantages of rapid and simple antibody production, controllable screening parameters and availability in generating a fully humanized antibody for treatment. Therefore, high-affinity and high-specificity antibodies suitable for the desired application can be easily engineered by these techniques, and phage display is now a major technological platform for the generation of candidate therapeutic antibodies.

The success of in vitro antibody generation technique largely depends on the quality and size of an antibody library. For phage and yeast display libraries (two most commonly-used methods), the size of the library depends on the transformation efficiency of host cells. Furthermore, various factors may affect the quality of the library, especially in a synthetic antibody library. Natural antibody library (Sheets MD et al. (1998) Proc. Nat'l Acad. Sci. USA 95, 6157-6162; Schwimmer L J et al. (2013) J. Immunol. Methods 391, 60-71) is obtained by PCR-amplifying V(D)J recombinant immunoglobulin genes from cDNA of B cells; therefore, there is no need for manual input to generate sequence differences. For synthetic antibody libraries, strategies for generating sequence differences are necessary, even critical. Sequence differences in most existing synthetic antibody libraries are concentrated in complementarity determining regions (CDRs), and obtained by random combinations of mononucleotide or trinucleotide units (Nissim Aet al. (1994) EMBO J, 13, 692-698; Tiller Tet al. (2013) MAbs 5, 445-470). CDR regions of a synthetic antibody library need to be designed to obtain a batch of amino acid sequences with large differences and distribution similar to the natural antibody of the host. A well-designed synthetic antibody library has several advantages, including high expression, good solubility, high stability, and easy to manipulate and optimize.

For synthetic antibody libraries, difference of libraries is realized by the addition of variable CDR regions to fixed framework regions. A suitable framework can provide a synthetic antibody library with the advantages of high stability, high expression, high compatibility with one another, being more suitable for human use, etc. (Arnaout Ret al. (2011) PLoS One 6, e22365). Many antibody libraries use, for example, DP47 and DPK22 as framework templates for construction (Silacci Met al. (2005) Proteomics 5, 2340-2350; Yang HY et al. (2009) Mol.

Cells 27, 225-235).

The easiest method to achieve difference of CDR regions is to synthesize random sequences with nucleotide mixtures. All 20 amino acids and stop codons can be encoded by NNK or NNS degenerate codons (N represents any base, K represents G or T, and S represents G or C). Other combinations of nucleotide mixtures can produce different sets of amino acids. For example, degenerate codons often used in CDR design include KMT (M represents A or C) encoding Ala, Asp, Ser or Tyr, WMC (W represents A or T) encoding Asn, Ser, Thr or Tyr, and RRT encoding Asn, Asp, Gly or Ser (R represents A or G). These degenerate codons are relatively easy and cost-effective to design, and some antibody libraries that are very useful are designed by this method (Yang HY et al. (2009) Mol. Cells 27, 225-235). The biggest disadvantage of this method is that the control accuracy of sequence differences is very low, and the random degenerate codon method can only allow an amino acid corresponding to a codon in the same row or column in a codon table. However, the trinucleotide-directed mutagenesis (TRIM) can freely insert any desired codon combination at the desired position. For TRIM, a pre-synthesized set of trinucleotide codons is used to synthesize differentiated CDRs (Prassler Jet al. (2011) J. Mol. Biol. 413, 261-278). By using a mixture of oligonucleotide synthesis units, a user can insert the desired combination of amino acids at any position in any distribution ratio. Therefore, CDRs can be designed to be closer to a natural amino acid distribution combination, and closer to a natural antibody.

Since antibody sequences do not have a fixed length, numbering antibody sequences first is a common step in antibody sequence analysis (Dunbar J and Deane CM (2016) Bioinformatics 32 (2), 298-300). Numbering antibody sequences is helpful for aligning positions with similar functions and spatial positions in an antibody, which can facilitate the division of regions on the antibody. For example, for an antibody heavy chain, positions 31-35 correspond to a CDR1 region.

High-throughput synthesis technique can simultaneously synthesize tens of thousands or even hundreds of thousands of nucleotide sequences longer than 100 bp on a single chip (Sriram K and George C (2014) Nat. Methods 11, 499-507), which enables to simultaneously synthesize all sets of nucleotide sequences required for CDR region differentiation. However, different from the synthesis methods as mentioned above, the chip synthesis is incapable of directly controlling the ratio of amino acids at each position by means of adjusting the ratio of the mixture. Therefore, the design scheme of CDR region synthesis is particularly critical. A suitable design scheme can both maximize the sequence variability and ensure to satisfy the given amino acid distribution ratio at each position.

### SUMMARY OF THE INVENTION

### 1) Technical Problems to be Solved

As mentioned above, when synthetic antibody libraries are constructed, a fixed frame plus diversity CDR library are used. Differences in antibody sequences are achieved based on differences in CDR libraries. In addition, in order to be close to the original natural antibody repertoire or a set of natural antibodies against a specific target, it is also desirable that each position of the CDR can be controlled to satisfy a specific amino acid distribution ratio (for example, the amino acid at position H32 satisfies the distribution ratio of 22% Ala, 32% Tyr and 46% Ser). When the high-throughput gene synthesis method, namely, the way for a large scale synthesis of CDR coding sequences is used, it is necessary to ensure the diversity of CDR regions (usually 10-100 possible amino acid sequences). At this point, determining the number for each possible amino acid sequence that can ensure a specific amino acid distribution ratio at each position becomes a problem to be solved. An object of the present invention is to provide a method for generating a CDR library by the high-throughput gene synthesis method, which simultaneously satisfies the CDR diversity and the specific amino acid distribution at each position.

### 2) Technical Solution

The methods of the present invention can comprise one or more of the following steps: step 1, according to requirements, listing all optional CDR amino acid sequences to form an alternative sequence set, wherein the number of alternative sequences is set as N, for example, if a certain CDR region consists of 5 amino acids, wherein position 1 is one of Asn, Asp, and Ser, position 2 is Tyr, position 3 is Gly, and position 4 is one of Ile and Met, and position 5 is His, then there are N=3×1×1×2×1=6 kinds of optional amino acid sequences; step 2, according to requirements, setting the total number of sequences (i.e. capacity) of a CDR library to M, wherein M is much larger than N, and then, according to the ratio of each amino acid at each position, calculating the number of sequences using the amino acid at the position; step 3, randomly selecting a sequence from an alternative sequence set, and judging whether the addition of the sequence to a library will cause the number of corresponding amino acids at a certain position to exceed the number calculated above, wherein if the number of corresponding amino acids does not exceed the number calculated above, then the sequence is added to the library, whereas if the number of corresponding amino acids exceeds the number calculated above, then the sequence is not added to the library and removed from the alternative sequence set; the number of sequences added to the library is set as L, and L and M are compared each time a sequence is added; cycling same if L<M, which indicates that the selection and storage have not been completed, and stopping same if L=M, which indicates that the selection and storage have been completed; step 4, reverse-translating the amino acid sequences in the library into DNA sequences, wherein identical amino acid sequences in the library can be translated simultaneously into DNA sequences; and step 5, performing subsequent high-throughput gene synthesis.

In some cases, for example, in the case that there are additional constraints on alternative sequences in addition to the specific distribution of various amino acids at various positions, such as excluding specific sequences, it is possible that after several rounds of selection (removal), all the amino acid sequences in the alternative set have been removed, but the library does not have enough sequences added. At this point, it is only necessary to randomly select sequences from the initial alternative set and add same to the library until L=M.

In the case that the library capacity M is very large, such as M=10⁶, step 3 will take a long time to perform the cycle. At this point, we can select a relatively smaller M′, such as M′=10⁴, and then expand to M. For example, the M′ library can be generated first according to steps 1 to 3 mentioned above; the probability distribution of amino acid sequences in the M′ library can be determined; sequences can be randomly selected from the initial alternative set according to the probability distribution; and finally generating the M library.

### 3) Beneficial Effects

The present invention realizes generating a CDR library by the high-throughput gene synthesis method, while simultaneously satisfies the CDR sequence diversity and the specific amino acid distribution at each position. The difference between the actual amino acid distribution ratio and the desired amino acid distribution ratio is within an acceptable range (e.g., within 1%). Moreover, since the step of randomly selecting sequences is used in the method of the present invention, the CDR library generated by the method of the present invention satisfies the CDR sequence diversity and the specific amino acid distribution at each position, has the advantage of random sequence distribution, and better mimics the sequence distribution of a natural antibody repertoire, which avoids or mitigates effects of human intervention. In addition, the present invention realizes high-precision control of the library construction.

### DETAILED DESCRIPTION OF EMBODIMENTS

In a first aspect, the present invention relates to a method for generating a CDR amino acid sequence library, comprising the steps of:

1. determining all allowable CDR amino acid sequences according to a predetermined type of an allowable amino acid residue at each position of a CDR amino acid sequence, and optionally according to a specified excluded sequence, to produce a set of alternative CDR amino acid sequences;

2. determining an allowable number for each amino acid residue at each position of the CDR amino acid sequence in the CDR amino acid sequence library according to a predetermined capacity of the CDR amino acid sequence library and a predetermined occurrence frequency of each allowable amino acid residue at each position of the CDR amino acid sequence; and

3. randomly selecting a CDR amino acid sequence from the set of alternative CDR amino acid sequences and adding the selected CDR amino acid sequence to the CDR amino acid sequence library and cycling same, until the cumulative number of CDR amino acid sequences added to the CDR amino acid sequence library reaches the predetermined capacity of the CDR amino acid sequence library, thereby generating the CDR amino acid sequence library, wherein
3.1 when the set of alternative CDR amino acid sequences has not been emptied, a CDR amino acid sequence is randomly selected from the set of alternative CDR amino acid sequences, wherein
   3.1.1 when adding the selected CDR amino acid sequence to the CDR amino acid sequence library does not result in that the cumulative number of corresponding amino acid residues at any position exceeds the allowable number of the corresponding amino acid residues at the position, adding the selected CDR amino acid sequence to the CDR amino acid sequence library, or
   3.1.2 when adding the selected CDR amino acid sequence to the CDR amino acid sequence library results in that the cumulative number of corresponding amino acid residues at at least one position exceeds the allowable number of the corresponding amino acid residues at the position, removing the selected CDR amino acid sequence from the set of alternative CDR amino acid sequences, or
3.2 when the set of alternative CDR amino acid sequences has been emptied, a CDR amino acid sequence is randomly selected from the initial set of alternative CDR amino acid sequences.

In a second aspect, the present invention relates to a method for generating a CDR nucleotide sequence library, comprising the steps of:

1. determining all allowable CDR amino acid sequences according to a predetermined type of an allowable amino acid residue at each position of a CDR amino acid sequence, and optionally according to a specified excluded sequence, to produce a set of alternative CDR amino acid sequences;

2. determining an allowable number for each amino acid residue at each position of the CDR amino acid sequence in the CDR amino acid sequence library according to a predetermined capacity of the CDR amino acid sequence library and a predetermined occurrence frequency of each allowable amino acid residue at each position of the CDR amino acid sequence;

3. randomly selecting a CDR amino acid sequence from the set of alternative CDR amino acid sequences and adding the selected CDR amino acid sequence to the CDR amino acid sequence library and cycling same, until the cumulative number of CDR amino acid sequences added to the CDR amino acid sequence library reaches the predetermined capacity of the CDR amino acid sequence library, thereby generating the CDR amino acid sequence library, wherein
3.1 when the set of alternative CDR amino acid sequences has not been emptied, a CDR amino acid sequence is randomly selected from the set of alternative CDR amino acid sequences, wherein
   3.1.1 when adding the selected CDR amino acid sequence to the CDR amino acid sequence library does not result in that the cumulative number of corresponding amino acid residues at any position exceeds the allowable number of the corresponding amino acid residues at the position, adding the selected CDR amino acid sequence to the CDR amino acid sequence library, or
   3.1.2 when adding the selected CDR amino acid sequence to the CDR amino acid sequence library results in that the cumulative number of corresponding amino acid residues at at least one position exceeds the allowable number of the corresponding amino acid residues at the position, removing the selected CDR amino acid sequence from the set of alternative CDR amino acid sequences, or
3.2 when the set of alternative CDR amino acid sequences has been emptied, a CDR amino acid sequence is randomly selected from the initial set of alternative CDR amino acid sequences; and

4. reverse-translating all the CDR amino acid sequences in the CDR amino acid sequence library into CDR nucleotide sequences, thereby generating the CDR nucleotide sequence library.

In a third aspect, the present invention relates to a method for generating a CDR nucleic acid library, comprising the steps of:

1. determining all allowable CDR amino acid sequences according to a predetermined type of an allowable amino acid residue at each position of a CDR amino acid sequence, and optionally according to a specified excluded sequence, to produce a set of alternative CDR amino acid sequences;

2. determining an allowable number for each amino acid residue at each position of the CDR amino acid sequence in the CDR amino acid sequence library according to a predetermined capacity of the CDR amino acid sequence library and a predetermined occurrence frequency of each allowable amino acid residue at each position of the CDR amino acid sequence;

3. randomly selecting a CDR amino acid sequence from the set of alternative CDR amino acid sequences and adding the selected CDR amino acid sequence to the CDR amino acid sequence library and cycling same, until the cumulative number of CDR amino acid sequences added to the CDR amino acid sequence library reaches the predetermined capacity of the CDR amino acid sequence library, thereby generating the CDR amino acid sequence library, wherein
3.1 when the set of alternative CDR amino acid sequences has not been emptied, a CDR amino acid sequence is randomly selected from the set of alternative CDR amino acid sequences, wherein
   3.1.1 when adding the selected CDR amino acid sequence to the CDR amino acid sequence library does not result in that the cumulative number of corresponding amino acid residues at any position exceeds the allowable number of the corresponding amino acid residues at the position, adding the selected CDR amino acid sequence to the CDR amino acid sequence library, or
   3.1.2 when adding the selected CDR amino acid sequence to the CDR amino acid sequence library results in that the cumulative number of corresponding amino acid residues at at least one position exceeds the allowable number of the corresponding amino acid residues at the position, removing the selected CDR amino acid sequence from the set of alternative CDR amino acid sequences, or
3.2 when the set of alternative CDR amino acid sequences has been emptied, a CDR amino acid sequence is randomly selected from the initial set of alternative CDR amino acid sequences;

4. reverse-translating all the CDR amino acid sequences in the CDR amino acid sequence library into CDR nucleotide sequences, thereby generating the CDR nucleotide sequence library; and

5. synthesizing CDR nucleic acids according to all the CDR nucleotide sequences in the CDR nucleotide sequence library, thereby generating the CDR nucleic acid library.

In a fourth aspect, the present invention relates to a method for generating a CDR peptide library, comprising the steps of:

1. determining all allowable CDR amino acid sequences according to a predetermined type of an allowable amino acid residue at each position of a CDR amino acid sequence, and optionally according to a specified excluded sequence, to produce a set of alternative CDR amino acid sequences;

2. determining an allowable number for each amino acid residue at each position of the CDR amino acid sequence in the CDR amino acid sequence library according to a predetermined capacity of the CDR amino acid sequence library and a predetermined occurrence frequency of each allowable amino acid residue at each position of the CDR amino acid sequence;

3. randomly selecting a CDR amino acid sequence from the set of alternative CDR amino acid sequences and adding the selected CDR amino acid sequence to the CDR amino acid sequence library and cycling same, until the cumulative number of CDR amino acid sequences added to the CDR amino acid sequence library reaches the predetermined capacity of the CDR amino acid sequence library, thereby generating the CDR amino acid sequence library, wherein
3.1 when the set of alternative CDR amino acid sequences has not been emptied, a CDR amino acid sequence is randomly selected from the set of alternative CDR amino acid sequences, wherein
   3.1.1 when adding the selected CDR amino acid sequence to the CDR amino acid sequence library does not result in that the cumulative number of corresponding amino acid residues at any position exceeds the allowable number of the corresponding amino acid residues at the position, adding the selected CDR amino acid sequence to the CDR amino acid sequence library, or
   3.1.2 when adding the selected CDR amino acid sequence to the CDR amino acid sequence library results in that the cumulative number of corresponding amino acid residues at at least one position exceeds the allowable number of the corresponding amino acid residues at the position, removing the selected CDR amino acid sequence from the set of alternative CDR amino acid sequences, or
3.2 when the set of alternative CDR amino acid sequences has been emptied, a CDR amino acid sequence is randomly selected from the initial set of alternative CDR amino acid sequences;

4. reverse-translating all the CDR amino acid sequences in the CDR amino acid sequence library into CDR nucleotide sequences, thereby generating the CDR nucleotide sequence library;

5. synthesizing CDR nucleic acids according to all the CDR nucleotide sequences in the CDR nucleotide sequence library, thereby generating the CDR nucleic acid library; and

6. expressing all the CDR nucleic acids in the CDR nucleic acid library in an expression system, thereby generating the CDR peptide library.

In a fifth aspect, the present invention relates to a method for generating a CDR peptide library, comprising the steps of:

1. determining all allowable CDR amino acid sequences according to a predetermined type of an allowable amino acid residue at each position of a CDR amino acid sequence, and optionally according to a specified excluded sequence, to produce a set of alternative CDR amino acid sequences;

2. determining an allowable number for each amino acid residue at each position of the CDR amino acid sequence in the CDR amino acid sequence library according to a predetermined capacity of the CDR amino acid sequence library and a predetermined occurrence frequency of each allowable amino acid residue at each position of the CDR amino acid sequence;

3. randomly selecting a CDR amino acid sequence from the set of alternative CDR amino acid sequences and adding the selected CDR amino acid sequence to the CDR amino acid sequence library and cycling same, until the cumulative number of CDR amino acid sequences added to the CDR amino acid sequence library reaches the predetermined capacity of the CDR amino acid sequence library, thereby generating the CDR amino acid sequence library, wherein
3.1 when the set of alternative CDR amino acid sequences has not been emptied, a CDR amino acid sequence is randomly selected from the set of alternative CDR amino acid sequences, wherein
   3.1.1 when adding the selected CDR amino acid sequence to the CDR amino acid sequence library does not result in that the cumulative number of corresponding amino acid residues at any position exceeds the allowable number of the corresponding amino acid residues at the position, adding the selected CDR amino acid sequence to the CDR amino acid sequence library, or
   3.1.2 when adding the selected CDR amino acid sequence to the CDR amino acid sequence library results in that the cumulative number of corresponding amino acid residues at at least one position exceeds the allowable number of the corresponding amino acid residues at the position, removing the selected CDR amino acid sequence from the set of alternative CDR amino acid sequences, or
3.2 when the set of alternative CDR amino acid sequences has been emptied, a CDR amino acid sequence is randomly selected from the initial set of alternative CDR amino acid sequences; and

4. synthesizing CDR peptides according to all the CDR amino acid sequences in the CDR amino acid sequence library, thereby generating the CDR peptide library.

In a sixth aspect, the present invention relates to a device for generating a CDR amino acid sequence library, comprising the following apparatus:
an input apparatus, which is configured for inputting a predetermined length of a CDR amino acid sequence, a predetermined type of an allowable amino acid residue and a predetermined occurrence frequency of each amino acid residue at each position of the CDR amino acid sequence, and/or a predetermined capacity of the CDR amino acid sequence library;
a processing apparatus, which is configured to be used for performing the operations of:

1. determining all allowable CDR amino acid sequences according to a predetermined type of an allowable amino acid residue at each position of a CDR amino acid sequence, and optionally according to a specified excluded sequence, to produce a set of alternative CDR amino acid sequences;

2. determining an allowable number for each amino acid residue at each position of the CDR amino acid sequence in the CDR amino acid sequence library according to a predetermined capacity of the CDR amino acid sequence library and a predetermined occurrence frequency of each allowable amino acid residue at each position of the CDR amino acid sequence;

3. randomly selecting a CDR amino acid sequence from the set of alternative CDR amino acid sequences and adding the selected CDR amino acid sequence to the CDR amino acid sequence library and cycling same, until the cumulative number of CDR amino acid sequences added to the CDR amino acid sequence library reaches the predetermined capacity of the CDR amino acid sequence library, thereby generating the CDR amino acid sequence library, wherein
3.1 when the set of alternative CDR amino acid sequences has not been emptied, a CDR amino acid sequence is randomly selected from the set of alternative CDR amino acid sequences, wherein
   3.1.1 when adding the selected CDR amino acid sequence to the CDR amino acid sequence library does not result in that the cumulative number of corresponding amino acid residues at any position exceeds the allowable number of the corresponding amino acid residues at the position, adding the selected CDR amino acid sequence to the CDR amino acid sequence library, or
   3.1.2 when adding the selected CDR amino acid sequence to the CDR amino acid sequence library results in that the cumulative number of corresponding amino acid residues at at least one position exceeds the allowable number of the corresponding amino acid residues at the position, removing the selected CDR amino acid sequence from the set of alternative CDR amino acid sequences, or
3.2 when the set of alternative CDR amino acid sequences has been emptied, a CDR amino acid sequence is randomly selected from the initial set of alternative CDR amino acid sequences; and
an output apparatus, which is configured for outputting the CDR amino acid sequence library.

In a seventh aspect, the present invention relates to a device for generating a CDR nucleotide sequence library, comprising the following apparatus:
an input apparatus, which is configured for inputting a predetermined length of a CDR amino acid sequence, a predetermined type of an allowable amino acid residue and a predetermined occurrence frequency of each amino acid residue at each position of the CDR amino acid sequence, and/or a predetermined capacity of the CDR amino acid sequence library;
a processing apparatus, which is configured to be used for performing the operations of:

1. determining all allowable CDR amino acid sequences according to a predetermined type of an allowable amino acid residue at each position of a CDR amino acid sequence, and optionally according to a specified excluded sequence, to produce a set of alternative CDR amino acid sequences;

2. determining an allowable number for each amino acid residue at each position of the CDR amino acid sequence in the CDR amino acid sequence library according to a predetermined capacity of the CDR amino acid sequence library and a predetermined occurrence frequency of each allowable amino acid residue at each position of the CDR amino acid sequence;

3. randomly selecting a CDR amino acid sequence from the set of alternative CDR amino acid sequences and adding the selected CDR amino acid sequence to the CDR amino acid sequence library and cycling same, until the cumulative number of CDR amino acid sequences added to the CDR amino acid sequence library reaches the predetermined capacity of the CDR amino acid sequence library, thereby generating the CDR amino acid sequence library, wherein
3.1 when the set of alternative CDR amino acid sequences has not been emptied, a CDR amino acid sequence is randomly selected from the set of alternative CDR amino acid sequences, wherein
   3.1.1 when adding the selected CDR amino acid sequence to the CDR amino acid sequence library does not result in that the cumulative number of corresponding amino acid residues at any position exceeds the allowable number of the corresponding amino acid residues at the position, adding the selected CDR amino acid sequence to the CDR amino acid sequence library, or
   3.1.2 when adding the selected CDR amino acid sequence to the CDR amino acid sequence library results in that the cumulative number of corresponding amino acid residues at at least one position exceeds the allowable number of the corresponding amino acid residues at the position, removing the selected CDR amino acid sequence from the set of alternative CDR amino acid sequences, or
3.2 when the set of alternative CDR amino acid sequences has been emptied, a CDR amino acid sequence is randomly selected from the initial set of alternative CDR amino acid sequences; and

4. reverse-translating all the CDR amino acid sequences in the CDR amino acid sequence library into CDR nucleotide sequences, thereby generating the CDR nucleotide sequence library; and
an output apparatus, which is configured for outputting the CDR nucleotide sequence library.

In an eighth aspect, the present invention relates to a device for generating a CDR nucleic acid library, comprising the following apparatus:
an input apparatus, which is configured for inputting a predetermined length of a CDR amino acid sequence, a predetermined type of an allowable amino acid residue and a predetermined occurrence frequency of each amino acid residue at each position of the CDR amino acid sequence, and/or a predetermined capacity of the CDR amino acid sequence library;
a processing apparatus, which is configured to be used for performing the operations of:

1. determining all allowable CDR amino acid sequences according to a predetermined type of an allowable amino acid residue at each position of a CDR amino acid sequence, and optionally according to a specified excluded sequence, to produce a set of alternative CDR amino acid sequences;

2. determining an allowable number for each amino acid residue at each position of the CDR amino acid sequence in the CDR amino acid sequence library according to a predetermined capacity of the CDR amino acid sequence library and a predetermined occurrence frequency of each allowable amino acid residue at each position of the CDR amino acid sequence;

3. randomly selecting a CDR amino acid sequence from the set of alternative CDR amino acid sequences and adding the selected CDR amino acid sequence to the CDR amino acid sequence library and cycling same, until the cumulative number of CDR amino acid sequences added to the CDR amino acid sequence library reaches the predetermined capacity of the CDR amino acid sequence library, thereby generating the CDR amino acid sequence library, wherein
3.1 when the set of alternative CDR amino acid sequences has not been emptied, a CDR amino acid sequence is randomly selected from the set of alternative CDR amino acid sequences, wherein
   3.1.1 when adding the selected CDR amino acid sequence to the CDR amino acid sequence library does not result in that the cumulative number of corresponding amino acid residues at any position exceeds the allowable number of the corresponding amino acid residues at the position, adding the selected CDR amino acid sequence to the CDR amino acid sequence library, or
   3.1.2 when adding the selected CDR amino acid sequence to the CDR amino acid sequence library results in that the cumulative number of corresponding amino acid residues at at least one position exceeds the allowable number of the corresponding amino acid residues at the position, removing the selected CDR amino acid sequence from the set of alternative CDR amino acid sequences, or
3.2 when the set of alternative CDR amino acid sequences has been emptied, a CDR amino acid sequence is randomly selected from the initial set of alternative CDR amino acid sequences; and

4. reverse-translating all the CDR amino acid sequences in the CDR amino acid sequence library into CDR nucleotide sequences, thereby generating the CDR nucleotide sequence library;
an output apparatus, which is configured for outputting the CDR nucleotide sequence library; and
a nucleic acid synthesis apparatus, which is configured for synthesizing CDR nucleic acids according to all the CDR nucleotide sequences in the CDR nucleotide sequence library, thereby generating the CDR nucleic acid library.

In a ninth aspect, the present invention relates to a device for generating a CDR peptide library, comprising the following apparatus:
an input apparatus, which is configured for inputting a predetermined length of a CDR amino acid sequence, a predetermined type of an allowable amino acid residue and a predetermined occurrence frequency of each amino acid residue at each position of the CDR amino acid sequence, and/or a predetermined capacity of the CDR amino acid sequence library;
a processing apparatus, which is configured to be used for performing the operations of:

1. determining all allowable CDR amino acid sequences according to a predetermined type of an allowable amino acid residue at each position of a CDR amino acid sequence, and optionally according to a specified excluded sequence, to produce a set of alternative CDR amino acid sequences;

2. determining an allowable number for each amino acid residue at each position of the CDR amino acid sequence in the CDR amino acid sequence library according to a predetermined capacity of the CDR amino acid sequence library and a predetermined occurrence frequency of each allowable amino acid residue at each position of the CDR amino acid sequence;

3. randomly selecting a CDR amino acid sequence from the set of alternative CDR amino acid sequences and adding the selected CDR amino acid sequence to the CDR amino acid sequence library and cycling same, until the cumulative number of CDR amino acid sequences added to the CDR amino acid sequence library reaches the predetermined capacity of the CDR amino acid sequence library, thereby generating the CDR amino acid sequence library, wherein
3.1 when the set of alternative CDR amino acid sequences has not been emptied, a CDR amino acid sequence is randomly selected from the set of alternative CDR amino acid sequences, wherein
   3.1.1 when adding the selected CDR amino acid sequence to the CDR amino acid sequence library does not result in that the cumulative number of corresponding amino acid residues at any position exceeds the allowable number of the corresponding amino acid residues at the position, adding the selected CDR amino acid sequence to the CDR amino acid sequence library, or
   3.1.2 when adding the selected CDR amino acid sequence to the CDR amino acid sequence library results in that the cumulative number of corresponding amino acid residues at at least one position exceeds the allowable number of the corresponding amino acid residues at the position, removing the selected CDR amino acid sequence from the set of alternative CDR amino acid sequences, or
   3.2 when the set of alternative CDR amino acid sequences has been emptied, a CDR amino acid sequence is randomly selected from the initial set of alternative CDR amino acid sequences; and

4. reverse-translating all the CDR amino acid sequences in the CDR amino acid sequence library into CDR nucleotide sequences, thereby generating the CDR nucleotide sequence library;
an output apparatus, which is configured for outputting the CDR nucleotide sequence library;
a nucleic acid synthesis apparatus, which is configured for synthesizing CDR nucleic acids according to all the CDR nucleotide sequences in the CDR nucleotide sequence library, thereby generating the CDR nucleic acid library; and
a peptide expression apparatus, which is configured for expressing all the CDR nucleic acids in the CDR nucleic acid library in an expression system, thereby generating the CDR peptide library.

In a tenth aspect, the present invention relates to a device for generating a CDR peptide library, comprising the following apparatus:
an input apparatus, which is configured for inputting a predetermined length of a CDR amino acid sequence, a predetermined type of an allowable amino acid residue and a predetermined occurrence frequency of each amino acid residue at each position of the CDR amino acid sequence, and/or a predetermined capacity of the CDR amino acid sequence library;
a processing apparatus, which is configured to be used for performing the operations of:

1. determining all allowable CDR amino acid sequences according to a predetermined type of an allowable amino acid residue at each position of a CDR amino acid sequence, and optionally according to a specified excluded sequence, to produce a set of alternative CDR amino acid sequences;

2. determining an allowable number for each amino acid residue at each position of the CDR amino acid sequence in the CDR amino acid sequence library according to a predetermined capacity of the CDR amino acid sequence library and a predetermined occurrence frequency of each allowable amino acid residue at each position of the CDR amino acid sequence; and

3. randomly selecting a CDR amino acid sequence from the set of alternative CDR amino acid sequences and adding the selected CDR amino acid sequence to the CDR amino acid sequence library and cycling same, until the cumulative number of CDR amino acid sequences added to the CDR amino acid sequence library reaches the predetermined capacity of the CDR amino acid sequence library, thereby generating the CDR amino acid sequence library, wherein
3.1 when the set of alternative CDR amino acid sequences has not been emptied, a CDR amino acid sequence is randomly selected from the set of alternative CDR amino acid sequences, wherein
   3.1.1 when adding the selected CDR amino acid sequence to the CDR amino acid sequence library does not result in that the cumulative number of corresponding amino acid residues at any position exceeds the allowable number of the corresponding amino acid residues at the position, adding the selected CDR amino acid sequence to the CDR amino acid sequence library, or
   3.1.2 when adding the selected CDR amino acid sequence to the CDR amino acid sequence library results in that the cumulative number of corresponding amino acid residues at at least one position exceeds the allowable number of the corresponding amino acid residues at the position, removing the selected CDR amino acid sequence from the set of alternative CDR amino acid sequences, or
3.2 when the set of alternative CDR amino acid sequences has been emptied, a CDR amino acid sequence is randomly selected from the initial set of alternative CDR amino acid sequences;
an output apparatus, which is configured for outputting the CDR amino acid sequence library; and
a peptide synthesis apparatus, which is configured for synthesizing CDR peptides according to all the CDR amino acid sequences in the CDR amino acid sequence library, thereby generating the CDR peptide library.

In one embodiment, the predetermined capacity of the CDR amino acid sequence library is 1,000 to 100,000 amino acid sequences, for example 1,000 to 90,000, 1,000 to 80,000, 1,000 to 75,000, 1,000 to 70,000, 1,000 to 60,000, 1,000 to 50,000, 1,000 to 40,000, 1,000 to 30,000, 1,000 to 25,000, 1,000 to 20,000, 1,000 to 10,000, 2,000 to 100,000, 2,500 to 100,000, 3,000 to 100,000, 4,000 to 100,000, 5,000 to 100,000, 6,000 to 100,000, 7,000 to 100,000, 7,500 to 100,000, 8,000 to 100,000, 9,000 to 100,000, or 10,000 to 100,000 amino acid sequences, for example 1,000, 2,000, 2,500, 3,000, 4,000, 5,000, 6,000, 7,000, 7,500, 8,000, 9,000, 10,000, 20,000, 25,000, 30,000, 40,000, 50,000, 60,000, 70,000, 75,000, 80,000, 90,000, 100,000 amino acid sequences.

In an eleventh aspect, the present invention relates to a method for generating a large CDR amino acid sequence library, comprising the steps of:

1. determining all allowable CDR amino acid sequences according to a predetermined type of an allowable amino acid residue at each position of a CDR amino acid sequence, and optionally according to a specified excluded sequence, to produce a set of alternative CDR amino acid sequences;

2. determining an allowable number for each amino acid residue at each position of the CDR amino acid sequence in a primary CDR amino acid sequence library according to a predetermined capacity of the primary CDR amino acid sequence library and a predetermined occurrence frequency of each allowable amino acid residue at each position of the CDR amino acid sequence;

3. randomly selecting a CDR amino acid sequence from the set of alternative CDR amino acid sequences and adding the selected CDR amino acid sequence to the primary CDR amino acid sequence library, and cycling same until the cumulative number of CDR amino acid sequences added to the primary CDR amino acid sequence library reaches the predetermined capacity of the primary CDR amino acid sequence library, thereby generating the primary CDR amino acid sequence library, wherein
3.1 when the set of alternative CDR amino acid sequences has not been emptied, a CDR amino acid sequence is randomly selected from the set of alternative CDR amino acid sequences, wherein
   3.1.1 when adding the selected CDR amino acid sequence to the primary CDR amino acid sequence library does not result in that the cumulative number of corresponding amino acid residues at any position exceeds the allowable number of the corresponding amino acid residues at the position, adding the selected CDR amino acid sequence to the primary CDR amino acid sequence library, or
   3.1.2 when adding the selected CDR amino acid sequence to the primary CDR amino acid sequence library results in that the cumulative number of corresponding amino acid residues at at least one position exceeds the allowable number of the corresponding amino acid residues at the position, removing the selected CDR amino acid sequence from the set of alternative CDR amino acid sequences, or
3.2 when the set of alternative CDR amino acid sequences has been emptied, a CDR amino acid sequence is randomly selected from the initial set of alternative CDR amino acid sequences;

4. determining an occurrence frequency of each CDR amino acid sequence in the primary CDR amino acid sequence library; and

5. according to the occurrence frequency of each CDR amino acid sequence in the primary CDR amino acid sequence library, randomly selecting a sequence from the initial set of alternative CDR amino acid sequences and adding the selected sequence to a secondary CDR amino acid sequence library, until the cumulative number of CDR amino acid sequences added to the secondary CDR amino acid sequence library reaches the predetermined capacity of the secondary CDR amino acid sequence library, thereby generating the secondary CDR amino acid sequence library, and thereby generating the large CDR amino acid sequence library.

In a twelfth aspect, the present invention relates to a method for generating a large CDR nucleotide sequence library, comprising the steps of:

1. determining all allowable CDR amino acid sequences according to a predetermined type of an allowable amino acid residue at each position of a CDR amino acid sequence, and optionally according to a specified excluded sequence, to produce a set of alternative CDR amino acid sequences;

2. determining an allowable number for each amino acid residue at each position of the CDR amino acid sequence in a primary CDR amino acid sequence library according to a predetermined capacity of the primary CDR amino acid sequence library and a predetermined occurrence frequency of each allowable amino acid residue at each position of the CDR amino acid sequence;

3. randomly selecting a CDR amino acid sequence from the set of alternative CDR amino acid sequences and adding the selected CDR amino acid sequence to the primary CDR amino acid sequence library, and cycling same until the cumulative number of CDR amino acid sequences added to the primary CDR amino acid sequence library reaches the predetermined capacity of the primary CDR amino acid sequence library, thereby generating the primary CDR amino acid sequence library, wherein
3.1 when the set of alternative CDR amino acid sequences has not been emptied, a CDR amino acid sequence is randomly selected from the set of alternative CDR amino acid sequences, wherein
   3.1.1 when adding the selected CDR amino acid sequence to the primary CDR amino acid sequence library does not result in that the cumulative number of corresponding amino acid residues at any position exceeds the allowable number of the corresponding amino acid residues at the position, adding the selected CDR amino acid sequence to the primary CDR amino acid sequence library, or
   3.1.2 when adding the selected CDR amino acid sequence to the primary CDR amino acid sequence library results in that the cumulative number of corresponding amino acid residues at at least one position exceeds the allowable number of the corresponding amino acid residues at the position, removing the selected CDR amino acid sequence from the set of alternative CDR amino acid sequences, or
3.2 when the set of alternative CDR amino acid sequences has been emptied, a CDR amino acid sequence is randomly selected from the initial set of alternative CDR amino acid sequences;

4. determining an occurrence frequency of each CDR amino acid sequence in the primary CDR amino acid sequence library;

5. according to the occurrence frequency of each CDR amino acid sequence in the primary CDR amino acid sequence library, randomly selecting a sequence from the initial set of alternative CDR amino acid sequences and adding the selected sequence to a secondary CDR amino acid sequence library, until the cumulative number of CDR amino acid sequences added to the secondary CDR amino acid sequence library reaches the predetermined capacity of the secondary CDR amino acid sequence library, thereby generating the secondary CDR amino acid sequence library; and

6. reverse-translating all the CDR amino acid sequences in the secondary CDR amino acid sequence library into CDR nucleotide sequences, thereby generating the large CDR nucleotide sequence library.

In a thirteenth aspect, the present invention relates to a method for generating a large CDR nucleic acid library, comprising the steps of:

1. determining all allowable CDR amino acid sequences according to a predetermined type of an allowable amino acid residue at each position of a CDR amino acid sequence, and optionally according to a specified excluded sequence, to produce a set of alternative CDR amino acid sequences;

2. determining an allowable number for each amino acid residue at each position of the CDR amino acid sequence in a primary CDR amino acid sequence library according to a predetermined capacity of the primary CDR amino acid sequence library and a predetermined occurrence frequency of each allowable amino acid residue at each position of the CDR amino acid sequence;

3. randomly selecting a CDR amino acid sequence from the set of alternative CDR amino acid sequences and adding the selected CDR amino acid sequence to the primary CDR amino acid sequence library, and cycling same until the cumulative number of CDR amino acid sequences added to the primary CDR amino acid sequence library reaches the predetermined capacity of the primary CDR amino acid sequence library, thereby generating the primary CDR amino acid sequence library, wherein
3.1 when the set of alternative CDR amino acid sequences has not been emptied, a CDR amino acid sequence is randomly selected from the set of alternative CDR amino acid sequences, wherein
   3.1.1 when adding the selected CDR amino acid sequence to the primary CDR amino acid sequence library does not result in that the cumulative number of corresponding amino acid residues at any position exceeds the allowable number of the corresponding amino acid residues at the position, adding the selected CDR amino acid sequence to the primary CDR amino acid sequence library, or
   3.1.2 when adding the selected CDR amino acid sequence to the primary CDR amino acid sequence library results in that the cumulative number of corresponding amino acid residues at at least one position exceeds the allowable number of the corresponding amino acid residues at the position, removing the selected CDR amino acid sequence from the set of alternative CDR amino acid sequences, or
3.2 when the set of alternative CDR amino acid sequences has been emptied, a CDR amino acid sequence is randomly selected from the initial set of alternative CDR amino acid sequences;

4. determining an occurrence frequency of each CDR amino acid sequence in the primary CDR amino acid sequence library;

5. according to the occurrence frequency of each CDR amino acid sequence in the primary CDR amino acid sequence library, randomly selecting a sequence from the initial set of alternative CDR amino acid sequences and adding the selected sequence to a secondary CDR amino acid sequence library, until the cumulative number of CDR amino acid sequences added to the secondary CDR amino acid sequence library reaches the predetermined capacity of the secondary CDR amino acid sequence library, thereby generating the secondary CDR amino acid sequence library;

6. reverse-translating all the CDR amino acid sequences in the secondary CDR amino acid sequence library into CDR nucleotide sequences, thereby generating the CDR nucleotide sequence library; and

7. synthesizing CDR nucleic acids according to all the CDR nucleotide sequences in the CDR nucleotide sequence library, thereby generating the large CDR nucleic acid library.

In a fourteenth aspect, the present invention relates to a method for generating a large CDR peptide library, comprising the steps of:

1. determining all allowable CDR amino acid sequences according to a predetermined type of an allowable amino acid residue at each position of a CDR amino acid sequence, and optionally according to a specified excluded sequence, to produce a set of alternative CDR amino acid sequences;

2. determining an allowable number for each amino acid residue at each position of the CDR amino acid sequence in a primary CDR amino acid sequence library according to a predetermined capacity of the primary CDR amino acid sequence library and a predetermined occurrence frequency of each allowable amino acid residue at each position of the CDR amino acid sequence;

3. randomly selecting a CDR amino acid sequence from the set of alternative CDR amino acid sequences and adding the selected CDR amino acid sequence to the primary CDR amino acid sequence library, and cycling same until the cumulative number of CDR amino acid sequences added to the primary CDR amino acid sequence library reaches the predetermined capacity of the primary CDR amino acid sequence library, thereby generating the primary CDR amino acid sequence library, wherein
3.1 when the set of alternative CDR amino acid sequences has not been emptied, a CDR amino acid sequence is randomly selected from the set of alternative CDR amino acid sequences, wherein
   3.1.1 when adding the selected CDR amino acid sequence to the primary CDR amino acid sequence library does not result in that the cumulative number of corresponding amino acid residues at any position exceeds the allowable number of the corresponding amino acid residues at the position, adding the selected CDR amino acid sequence to the primary CDR amino acid sequence library, or
   3.1.2 when adding the selected CDR amino acid sequence to the primary CDR amino acid sequence library results in that the cumulative number of corresponding amino acid residues at at least one position exceeds the allowable number of the corresponding amino acid residues at the position, removing the selected CDR amino acid sequence from the set of alternative CDR amino acid sequences, or
3.2 when the set of alternative CDR amino acid sequences has been emptied, a CDR amino acid sequence is randomly selected from the initial set of alternative CDR amino acid sequences;

4. determining an occurrence frequency of each CDR amino acid sequence in the primary CDR amino acid sequence library;

5. according to the occurrence frequency of each CDR amino acid sequence in the primary CDR amino acid sequence library, randomly selecting a sequence from the initial set of alternative CDR amino acid sequences and adding the selected sequence to a secondary CDR amino acid sequence library, until the cumulative number of CDR amino acid sequences added to the secondary CDR amino acid sequence library reaches the predetermined capacity of the secondary CDR amino acid sequence library, thereby generating the secondary CDR amino acid sequence library;

6. reverse-translating all the CDR amino acid sequences in the secondary CDR amino acid sequence library into CDR nucleotide sequences, thereby generating the CDR nucleotide sequence library;

7. synthesizing CDR nucleic acids according to all the CDR nucleotide sequences in the CDR nucleotide sequence library, thereby generating the CDR nucleic acid library; and

8. expressing all the CDR nucleic acids in the CDR nucleic acid library in an expression system, thereby generating the large CDR peptide library.

In a fifteenth aspect, the present invention relates to a method for generating a large CDR peptide library, comprising the steps of:

1. determining all allowable CDR amino acid sequences according to a predetermined type of an allowable amino acid residue at each position of a CDR amino acid sequence, and optionally according to a specified excluded sequence, to produce a set of alternative CDR amino acid sequences;

2. determining an allowable number for each amino acid residue at each position of the CDR amino acid sequence in a primary CDR amino acid sequence library according to a predetermined capacity of the primary CDR amino acid sequence library and a predetermined occurrence frequency of each allowable amino acid residue at each position of the CDR amino acid sequence;

3. randomly selecting a CDR amino acid sequence from the set of alternative CDR amino acid sequences and adding the selected CDR amino acid sequence to the primary CDR amino acid sequence library, and cycling same until the cumulative number of CDR amino acid sequences added to the primary CDR amino acid sequence library reaches the predetermined capacity of the primary CDR amino acid sequence library, thereby generating the primary CDR amino acid sequence library, wherein
3.1 when the set of alternative CDR amino acid sequences has not been emptied, a CDR amino acid sequence is randomly selected from the set of alternative CDR amino acid sequences, wherein
   3.1.1 when adding the selected CDR amino acid sequence to the primary CDR amino acid sequence library does not result in that the cumulative number of corresponding amino acid residues at any position exceeds the allowable number of the corresponding amino acid residues at the position, adding the selected CDR amino acid sequence to the primary CDR amino acid sequence library, or
   3.1.2 when adding the selected CDR amino acid sequence to the primary CDR amino acid sequence library results in that the cumulative number of corresponding amino acid residues at at least one position exceeds the allowable number of the corresponding amino acid residues at the position, removing the selected CDR amino acid sequence from the set of alternative CDR amino acid sequences, or
3.2 when the set of alternative CDR amino acid sequences has been emptied, a CDR amino acid sequence is randomly selected from the initial set of alternative CDR amino acid sequences;

4. determining an occurrence frequency of each CDR amino acid sequence in the primary CDR amino acid sequence library;

5. according to the occurrence frequency of each CDR amino acid sequence in the primary CDR amino acid sequence library, randomly selecting a sequence from the initial set of alternative CDR amino acid sequences and adding the selected sequence to a secondary CDR amino acid sequence library, until the cumulative number of CDR amino acid sequences added to the secondary CDR amino acid sequence library reaches the predetermined capacity of the secondary CDR amino acid sequence library, thereby generating the secondary CDR amino acid sequence library; and

6. synthesizing CDR peptides according to all the CDR amino acid sequences in the CDR amino acid sequence library, thereby generating the large CDR peptide library.

In a sixteenth aspect, the present invention relates to a device for generating a large CDR amino acid sequence library, comprising the following apparatus:
an input apparatus, which is configured for inputting a predetermined length of a CDR amino acid sequence, a predetermined type of an allowable amino acid residue and a predetermined occurrence frequency of each amino acid residue at each position of the CDR amino acid sequence, a predetermined capacity of a primary CDR amino acid sequence library, and/or a predetermined capacity of a secondary CDR amino acid sequence library;
a processing apparatus, which is configured to be used for performing the operations of:

1. determining all allowable CDR amino acid sequences according to a predetermined type of an allowable amino acid residue at each position of a CDR amino acid sequence, and optionally according to a specified excluded sequence, to produce a set of alternative CDR amino acid sequences;

2. determining an allowable number for each amino acid residue at each position of the CDR amino acid sequence in a primary CDR amino acid sequence library according to a predetermined capacity of the primary CDR amino acid sequence library and a predetermined occurrence frequency of each allowable amino acid residue at each position of the CDR amino acid sequence;

3. randomly selecting a CDR amino acid sequence from the set of alternative CDR amino acid sequences and adding the selected CDR amino acid sequence to the primary CDR amino acid sequence library, and cycling same until the cumulative number of CDR amino acid sequences added to the primary CDR amino acid sequence library reaches the predetermined capacity of the primary CDR amino acid sequence library, thereby generating the primary CDR amino acid sequence library, wherein
3.1 when the set of alternative CDR amino acid sequences has not been emptied, a CDR amino acid sequence is randomly selected from the set of alternative CDR amino acid sequences, wherein
   3.1.1 when adding the selected CDR amino acid sequence to the primary CDR amino acid sequence library does not result in that the cumulative number of corresponding amino acid residues at any position exceeds the allowable number of the corresponding amino acid residues at the position, adding the selected CDR amino acid sequence to the primary CDR amino acid sequence library, or
   3.1.2 when adding the selected CDR amino acid sequence to the primary CDR amino acid sequence library results in that the cumulative number of corresponding amino acid residues at at least one position exceeds the allowable number of the corresponding amino acid residues at the position, removing the selected CDR amino acid sequence from the set of alternative CDR amino acid sequences, or
3.2 when the set of alternative CDR amino acid sequences has been emptied, a CDR amino acid sequence is randomly selected from the initial set of alternative CDR amino acid sequences;

4. determining an occurrence frequency of each CDR amino acid sequence in the primary CDR amino acid sequence library; and

5. according to the occurrence frequency of each CDR amino acid sequence in the primary CDR amino acid sequence library, randomly selecting a sequence from the initial set of alternative CDR amino acid sequences and adding the selected sequence to a secondary CDR amino acid sequence library, until the cumulative number of CDR amino acid sequences added to the secondary CDR amino acid sequence library reaches the predetermined capacity of the secondary CDR amino acid sequence library, thereby generating the secondary CDR amino acid sequence library, and thereby generating the large CDR amino acid sequence library; and
an output apparatus, which is configured for outputting the large CDR amino acid sequence library.

n a seventeenth aspect, the present invention relates to a device for generating a large CDR nucleotide sequence library, comprising the following apparatus:
an input apparatus, which is configured for inputting a predetermined length of a CDR amino acid sequence, a predetermined type of an allowable amino acid residue and a predetermined occurrence frequency of each amino acid residue at each position of the CDR amino acid sequence, a predetermined capacity of a primary CDR amino acid sequence library, and/or a predetermined capacity of a secondary CDR amino acid sequence library;
a processing apparatus, which is configured to be used for performing the operations of:

1. determining all allowable CDR amino acid sequences according to a predetermined type of an allowable amino acid residue at each position of a CDR amino acid sequence, and optionally according to a specified excluded sequence, to produce a set of alternative CDR amino acid sequences;

2. determining an allowable number for each amino acid residue at each position of the CDR amino acid sequence in a primary CDR amino acid sequence library according to a predetermined capacity of the primary CDR amino acid sequence library and a predetermined occurrence frequency of each allowable amino acid residue at each position of the CDR amino acid sequence;

3. randomly selecting a CDR amino acid sequence from the set of alternative CDR amino acid sequences and adding the selected CDR amino acid sequence to the primary CDR amino acid sequence library, and cycling same until the cumulative number of CDR amino acid sequences added to the primary CDR amino acid sequence library reaches the predetermined capacity of the primary CDR amino acid sequence library, thereby generating the primary CDR amino acid sequence library, wherein
3.1 when the set of alternative CDR amino acid sequences has not been emptied, a CDR amino acid sequence is randomly selected from the set of alternative CDR amino acid sequences, wherein
   3.1.1 when adding the selected CDR amino acid sequence to the primary CDR amino acid sequence library does not result in that the cumulative number of corresponding amino acid residues at any position exceeds the allowable number of the corresponding amino acid residues at the position, adding the selected CDR amino acid sequence to the primary CDR amino acid sequence library, or
   3.1.2 when adding the selected CDR amino acid sequence to the primary CDR amino acid sequence library results in that the cumulative number of corresponding amino acid residues at at least one position exceeds the allowable number of the corresponding amino acid residues at the position, removing the selected CDR amino acid sequence from the set of alternative CDR amino acid sequences, or
3.2 when the set of alternative CDR amino acid sequences has been emptied, a CDR amino acid sequence is randomly selected from the initial set of alternative CDR amino acid sequences;

4. determining an occurrence frequency of each CDR amino acid sequence in the primary CDR amino acid sequence library;

5. according to the occurrence frequency of each CDR amino acid sequence in the primary CDR amino acid sequence library, randomly selecting a sequence from the initial set of alternative CDR amino acid sequences and adding the selected sequence to a secondary CDR amino acid sequence library, until the cumulative number of CDR amino acid sequences added to the secondary CDR amino acid sequence library reaches the predetermined capacity of the secondary CDR amino acid sequence library, thereby generating the secondary CDR amino acid sequence library; and

6. reverse-translating all the CDR amino acid sequences in the secondary CDR amino acid sequence library into CDR nucleotide sequences, thereby generating the large CDR nucleotide sequence library; and
an output apparatus, which is configured for outputting the large CDR nucleotide sequence library.

In an eighteenth aspect, the present invention relates to a device for generating a large CDR nucleic acid library, comprising the following apparatus:
an input apparatus, which is configured for inputting a predetermined length of a CDR amino acid sequence, a predetermined type of an allowable amino acid residue and a predetermined occurrence frequency of each amino acid residue at each position of the CDR amino acid sequence, a predetermined capacity of a primary CDR amino acid sequence library, and/or a predetermined capacity of a secondary CDR amino acid sequence library;
a processing apparatus, which is configured to be used for performing the operations of:

1. determining all allowable CDR amino acid sequences according to a predetermined type of an allowable amino acid residue at each position of a CDR amino acid sequence, and optionally according to a specified excluded sequence, to produce a set of alternative CDR amino acid sequences;

2. determining an allowable number for each amino acid residue at each position of the CDR amino acid sequence in a primary CDR amino acid sequence library according to a predetermined capacity of the primary CDR amino acid sequence library and a predetermined occurrence frequency of each allowable amino acid residue at each position of the CDR amino acid sequence;

3. randomly selecting a CDR amino acid sequence from the set of alternative CDR amino acid sequences and adding the selected CDR amino acid sequence to the primary CDR amino acid sequence library, and cycling same until the cumulative number of CDR amino acid sequences added to the primary CDR amino acid sequence library reaches the predetermined capacity of the primary CDR amino acid sequence library, thereby generating the primary CDR amino acid sequence library, wherein
3.1 when the set of alternative CDR amino acid sequences has not been emptied, a CDR amino acid sequence is randomly selected from the set of alternative CDR amino acid sequences, wherein
   3.1.1 when adding the selected CDR amino acid sequence to the primary CDR amino acid sequence library does not result in that the cumulative number of corresponding amino acid residues at any position exceeds the allowable number of the corresponding amino acid residues at the position, adding the selected CDR amino acid sequence to the primary CDR amino acid sequence library, or
   3.1.2 when adding the selected CDR amino acid sequence to the primary CDR amino acid sequence library results in that the cumulative number of corresponding amino acid residues at at least one position exceeds the allowable number of the corresponding amino acid residues at the position, removing the selected CDR amino acid sequence from the set of alternative CDR amino acid sequences, or
3.2 when the set of alternative CDR amino acid sequences has been emptied, a CDR amino acid sequence is randomly selected from the initial set of alternative CDR amino acid sequences;

4. determining an occurrence frequency of each CDR amino acid sequence in the primary CDR amino acid sequence library;

5. according to the occurrence frequency of each CDR amino acid sequence in the primary CDR amino acid sequence library, randomly selecting a sequence from the initial set of alternative CDR amino acid sequences and adding the selected sequence to a secondary CDR amino acid sequence library, until the cumulative number of CDR amino acid sequences added to the secondary CDR amino acid sequence library reaches the predetermined capacity of the secondary CDR amino acid sequence library, thereby generating the secondary CDR amino acid sequence library; and

6. reverse-translating all the CDR amino acid sequences in the secondary CDR amino acid sequence library into CDR nucleotide sequences, thereby generating the CDR nucleotide sequence library;
an output apparatus, which is configured for outputting the CDR nucleotide sequence library; and
a nucleic acid synthesis apparatus, which is configured for synthesizing CDR nucleic acids according to all the CDR nucleotide sequences in the CDR nucleotide sequence library, thereby generating the large CDR nucleic acid library.

In a nineteenth aspect, the present invention relates to a device for generating a large CDR peptide library, comprising the following apparatus:
an input apparatus, which is configured for inputting a predetermined length of a CDR amino acid sequence, a predetermined type of an allowable amino acid residue and a predetermined occurrence frequency of each amino acid residue at each position of the CDR amino acid sequence, a predetermined capacity of a primary CDR amino acid sequence library, and/or a predetermined capacity of a secondary CDR amino acid sequence library;
a processing apparatus, which is configured to be used for performing the operations of:

1. determining all allowable CDR amino acid sequences according to a predetermined type of an allowable amino acid residue at each position of a CDR amino acid sequence, and optionally according to a specified excluded sequence, to produce a set of alternative CDR amino acid sequences;

2. determining an allowable number for each amino acid residue at each position of the CDR amino acid sequence in a primary CDR amino acid sequence library according to a predetermined capacity of the primary CDR amino acid sequence library and a predetermined occurrence frequency of each allowable amino acid residue at each position of the CDR amino acid sequence;

3. randomly selecting a CDR amino acid sequence from the set of alternative CDR amino acid sequences and adding the selected CDR amino acid sequence to the primary CDR amino acid sequence library, and cycling same until the cumulative number of CDR amino acid sequences added to the primary CDR amino acid sequence library reaches the predetermined capacity of the primary CDR amino acid sequence library, thereby generating the primary CDR amino acid sequence library, wherein
3.1 when the set of alternative CDR amino acid sequences has not been emptied, a CDR amino acid sequence is randomly selected from the set of alternative CDR amino acid sequences, wherein
   3.1.1 when adding the selected CDR amino acid sequence to the primary CDR amino acid sequence library does not result in that the cumulative number of corresponding amino acid residues at any position exceeds the allowable number of the corresponding amino acid residues at the position, adding the selected CDR amino acid sequence to the primary CDR amino acid sequence library, or
   3.1.2 when adding the selected CDR amino acid sequence to the primary CDR amino acid sequence library results in that the cumulative number of corresponding amino acid residues at at least one position exceeds the allowable number of the corresponding amino acid residues at the position, removing the selected CDR amino acid sequence from the set of alternative CDR amino acid sequences, or
3.2 when the set of alternative CDR amino acid sequences has been emptied, a CDR amino acid sequence is randomly selected from the initial set of alternative CDR amino acid sequences;

4. determining an occurrence frequency of each CDR amino acid sequence in the primary CDR amino acid sequence library;

5. according to the occurrence frequency of each CDR amino acid sequence in the primary CDR amino acid sequence library, randomly selecting a sequence from the initial set of alternative CDR amino acid sequences and adding the selected sequence to a secondary CDR amino acid sequence library, until the cumulative number of CDR amino acid sequences added to the secondary CDR amino acid sequence library reaches the predetermined capacity of the secondary CDR amino acid sequence library, thereby generating the secondary CDR amino acid sequence library; and

6. reverse-translating all the CDR amino acid sequences in the secondary CDR amino acid sequence library into CDR nucleotide sequences, thereby generating the CDR nucleotide sequence library;
an output apparatus, which is configured for outputting the CDR nucleotide sequence library;
a nucleic acid synthesis apparatus, which is configured for synthesizing CDR nucleic acids according to all the CDR nucleotide sequences in the CDR nucleotide sequence library, thereby generating the CDR nucleic acid library; and
a peptide expression apparatus, which is configured for expressing all the CDR nucleic acids in the CDR nucleic acid library in an expression system, thereby generating the large CDR peptide library.

In a twentieth aspect, the present invention relates to a device for generating a large CDR peptide library, comprising the following apparatus:
an input apparatus, which is configured for inputting a predetermined length of a CDR amino acid sequence, a predetermined type of an allowable amino acid residue and a predetermined occurrence frequency of each amino acid residue at each position of the CDR amino acid sequence, a predetermined capacity of a primary CDR amino acid sequence library, and/or a predetermined capacity of a secondary CDR amino acid sequence library;
a processing apparatus, which is configured to be used for performing the operations of:

1. determining all allowable CDR amino acid sequences according to a predetermined type of an allowable amino acid residue at each position of a CDR amino acid sequence, and optionally according to a specified excluded sequence, to produce a set of alternative CDR amino acid sequences;

2. determining an allowable number for each amino acid residue at each position of the CDR amino acid sequence in a primary CDR amino acid sequence library according to a predetermined capacity of the primary CDR amino acid sequence library and a predetermined occurrence frequency of each allowable amino acid residue at each position of the CDR amino acid sequence;

3. randomly selecting a CDR amino acid sequence from the set of alternative CDR amino acid sequences and adding the selected CDR amino acid sequence to the primary CDR amino acid sequence library, and cycling same until the cumulative number of CDR amino acid sequences added to the primary CDR amino acid sequence library reaches the predetermined capacity of the primary CDR amino acid sequence library, thereby generating the primary CDR amino acid sequence library, wherein
3.1 when the set of alternative CDR amino acid sequences has not been emptied, a CDR amino acid sequence is randomly selected from the set of alternative CDR amino acid sequences, wherein
   3.1.1 when adding the selected CDR amino acid sequence to the primary CDR amino acid sequence library does not result in that the cumulative number of corresponding amino acid residues at any position exceeds the allowable number of the corresponding amino acid residues at the position, adding the selected CDR amino acid sequence to the primary CDR amino acid sequence library, or
   3.1.2 when adding the selected CDR amino acid sequence to the primary CDR amino acid sequence library results in that the cumulative number of corresponding amino acid residues at at least one position exceeds the allowable number of the corresponding amino acid residues at the position, removing the selected CDR amino acid sequence from the set of alternative CDR amino acid sequences, or
3.2 when the set of alternative CDR amino acid sequences has been emptied, a CDR amino acid sequence is randomly selected from the initial set of alternative CDR amino acid sequences;

4. determining an occurrence frequency of each CDR amino acid sequence in the primary CDR amino acid sequence library; and

5. according to the occurrence frequency of each CDR amino acid sequence in the primary CDR amino acid sequence library, randomly selecting a sequence from the initial set of alternative CDR amino acid sequences and adding the selected sequence to a secondary CDR amino acid sequence library, until the cumulative number of CDR amino acid sequences added to the secondary CDR amino acid sequence library reaches the predetermined capacity of the secondary CDR amino acid sequence library, thereby generating the secondary CDR amino acid sequence library;
an output apparatus, which is configured for outputting the secondary CDR amino acid sequence library; and
a peptide expression apparatus, which is configured for synthesizing CDR peptides according to all the CDR amino acid sequences in the secondary CDR amino acid sequence library, thereby generating the large CDR peptide library.

In one embodiment, the predetermined capacity of the primary CDR amino acid sequence library is about 1,000 to 100,000 amino acid sequences, for example about 1,000 to 90,000, 1,000 to 80,000, 1,000 to 75,000, 1,000 to 70,000, 1,000 to 60,000, 1,000 to 50,000, 1,000 to 40,000, 1,000 to 30,000, 1,000 to 25,000, 1,000 to 20,000, 1,000 to 10,000, 2,000 to 100,000, 2,500 to 100,000, 3,000 to 100,000, 4,000 to 100,000, 5,000 to 100,000, 6,000 to 100,000, 7,000 to 100,000, 7,500 to 100,000, 8,000 to 100,000, 9,000 to 100,000, or 10,000 to 100,000 amino acid sequences, for example about 1,000, 2,000, 2,500, 3,000, 4,000, 5,000, 6,000, 7,000, 7,500, 8,000, 9,000, 10,000, 20,000, 25,000, 30,000, 40,000, 50,000, 60,000, 70,000, 75,000, 80,000, 90,000, 100,000 amino acid sequences.

In one embodiment, the predetermined capacity of the secondary CDR amino acid sequence library is about 1 to 10000 times or even more, for example, about 10 to 1000 times, 10 to 900 times, 10 to 800 times, 10 to 700 times, 10 to 600 times, 10 to 500 times, 10 to 400 times, 10 to 300 times, 10 to 200 times, 10 to 100 times, 10 to 90 times, 10 to 80 times, 10 to 70 times, 10 to 60 times, 10 to 50 times, 10 to 40 times, 10 to 30 times, 10 to 20 times, 20 to 1000 times, 30 to 1000 times, 40 to 1000 times, 50 to 1000 times, 60 to 1000 times, 70 to 1000 times, 80 to 1000 times, 90 to 1000 times, 100 to 1000 times, 200 to 1000 times, 300 to 1000 times, 400 to 1000 times, 500 to 1000 times, 600 to 1000 times, 700 to 1000 times, 800 to 1000 times, or 900 to 1000 times, for example, about 10, 20, 25, 30, 40, 50, 60, 70, 75, 80, 90, 100, 200, 250, 300, 400, 500, 600, 700, 750, 800, 900, or 1000 times the predetermined capacity of the primary CDR amino acid sequence library.

The device of the present invention can further comprise a storage apparatus, which is configured to store an algorithm for performing the operations.

In a twenty-first aspect, the present invention relates to a computer program product, comprising a computer program instruction, wherein when the instruction is executed by a computer, the above-mentioned method is implemented and/or the above-mentioned device is operated.

In a twenty-second aspect, the present invention relates to a storage apparatus, which stores the above-mentioned computer program product.

In one embodiment, the CDR is antibody heavy chain CDR1, CDR2 and/or CDR3, and/or light chain CDR1, CDR2 and/or CDR3. In one embodiment, the antibody is a mammalian antibody, e.g., a rodent antibody (e.g., a mouse, rat or rabbit antibody) or a primate antibody (e.g., a cynomolgus or human antibody). In one embodiment, the antibody is a human antibody, a humanized antibody, or a chimeric antibody.

The present invention can be used in, but not limited to an antibody CDR. In fact, the present invention can be used for any peptide (alternatively referred to as oligopeptide, polypeptide, protein, amino acid polymer, etc.) of interest in diversity. For example, the present invention can be used for the diversity of acting site of one or both of two molecules that interact (e.g., recognize, bind, modify, cleave, etc.), e.g., antibody-antigen, receptor-ligand and enzyme-substrate. Moreover, the present invention can also be used for other polymer molecules of interest in diversity, such as polysaccharide or nucleic acid, especially functional, non-coding nucleic acid, such as functional RNA.

In one embodiment, the predetermined length of the CDR amino acid sequence is about 3 to 20 or more amino acid residues, for example about 3 to 15, 3 to 10, 3 to 5, 5 to 20, 5 to 15, 5 to 10, 5 to 7, 10 to 20, 10 to 15, or 15 to 20 amino acid residues, for example about 3, 4, 5, 6, 7, 8, 9, 10, 11, 12, 13, 14, 15, 16, 17, 18, 19, or 20 amino acid residues. The length of CDR amino acid sequences in a CDR library are generally the same. However, the length of CDR amino acid sequences in a CDR library can be different, and in this case, “deletion” is provided as an option for amino acids at one or more positions.

As mentioned above, the present invention can be used in, but not limited to CDR, or even to peptide. Therefore, the above content is also suitable for other sequences, such as nucleotide sequences. Furthermore, the present invention can also be used in, but not limited to the above-mentioned sequence length. A person skilled in the art would appreciate that the sequence length has a small effect on the implementation of the present invention, and the sequence complexity (i.e., the number of variable positions and the number of types of alternative amino acid/nucleotide residues at each variable position) has a great effect on the implementation of the present invention. In other words, the sequence of the present invention can comprise 3 to 20 or more, for example about 3 to 15, 3 to 10, 3 to 5, 5 to 20, 5 to 15, 5 to 10, 5 to 7, 10 to 20, 10 to 15, or 15 to 20, for example 3, 4, 5, 6, 7, 8, 9, 10, 11, 12, 13, 14, 15, 16, 17, 18, 19, or 20 variable positions. In this case, the full length of the sequence can be longer. The full length of a sequence is mainly affected by the efficiency of a synthesizer to synthesize the sequence. Variable positions can be completely contiguous (i.e., all variable positions are connected into one segment), completely discontinuous (i.e., any two variable positions are not connected), or neither (i.e., some but not all variable positions are connected into one or more segments, and there may also be one or more isolated variable positions).

In one embodiment, each position allows selection of about 1 to 20 common amino acid residues, e.g., about 2 to 10, 3 to 10, or 5 to 10 common amino acid residues, e.g., about 1, 2, 3, 4, 5, 6, 7, 8, 9, 10, 11, 12, 13, 14, 15, 16, 17, 18, 19, or 20 common amino acid residues. In one embodiment, the number of types of amino acid residues allowed to be selected at each position is identical. In one embodiment, the number of types of amino acid residues allowed to be selected at each position is different. In one embodiment, the number of types of amino acid residues allowed to be selected at some positions is identical. In one embodiment, the number of types of amino acid residues allowed to be selected at some positions is different. In one embodiment, the number of types of amino acid residues allowed to be selected at all positions is identical. In one embodiment, the number of types of amino acid residues allowed to be selected at all positions is different. The present invention can be used in, but not limited to the 20 common amino acids, and can also be used in all known amino acids, especially in chemically synthesized peptide libraries.

As mentioned above, the present invention can be used in, but not limited to peptide, but can also be used in other polymer molecules. Therefore, the above content is also suitable for other building blocks such as nucleotide and monosaccharide.

In one embodiment, the (initial) set of alternative CDR amino acid sequences comprises about 10 to 1000 allowable CDR amino acid sequences, for example about 10 to 900, 10 to 800, 10 to 750, 10 to 700, 10 to 600, 10 to 500, 10 to 400, 10 to 300, 10 to 250, 10 to 200, 10 to 100, 10 to 90, 10 to 80, 10 to 75, 10 to 70, 10 to 60, 10 to 50, 10 to 40, 10 to 30, 10 to 25, 10 to 20, 20 to 1000, 25 to 1000, 30 to 1000, 40 to 1000, 50 to 1000, 60 to 1000, 70 to 1000, 75 to 1000, 80 to 1000, 90 to 1000, 100 to 1000, 200 to 1000, 250 to 1000, 300 to 1000, 400 to 1000, 500 to 1000, 600 to 1000, 700 to 1000, 750 to 1000, 800 to 1000, or 900 to 1000, for example about 10, 20, 25, 30, 40, 50, 60, 70, 75, 80, 90, 100, 200, 250, 300, 400, 500, 600, 700, 750, 800, 900, or 1000 allowable CDR amino acid sequences.

Generally, an amino acid sequence in a library is encoded by a nucleotide sequence (DNA sequence (in the case of expression using intracellular translation) or RNA sequence (in the case of expression using extracellular translation)). In this case, reverse translation is usually performed using codons that are unique or preferred (or most frequently occurring in nature) by the host cell or expression system. Alternatively, an amino acid sequence in a library can be encoded by multiple nucleotide sequences (e.g., due to codon redundancy). In this case, the capacity of the nucleotide sequence library may be larger than the capacity of the amino acid sequence library.

The methods for randomly selecting a sequence from an alternative sequence set are well known in the art. For example, the interval [0, 1] can be divided into n intervals in equal proportions according to the number n of the sequences in the alternative sequence set, and each interval corresponds to a sequence. A random number generator is then used to generate the number x {x∈R|0≤x≤1} according to the average distribution. The corresponding sequence is selected according to the subinterval to which x belongs. For another example, the choice function in random submodule of numpy module of the python software can be used, wherein parameter a is set as the set of alternative sequences.

The methods for randomly selecting a sequence from an (initial) set of alternative sequences in proportion (e.g., according to the occurrence frequency of each sequence in a primary library) are also well known in the art. For example, the interval [0, 1] can be divided into n intervals according to the number n of the sequences in the alternative sequence set, and each interval corresponds to a sequence. The size of each interval is proportional to its corresponding selection probability (i.e., the above-mentioned occurrence frequency). A random number generator is then used to generate the number x {x∈R|0≤x≤1} according to the average distribution. The corresponding sequence is selected according to the subinterval to which x belongs. For another example, the choice function in random submodule of numpy module of the python software can be used, wherein the parameter a is set as the set of alternative sequences, and parameter p is set as the selection probability of each sequence in the alternative sequence set (e.g., the occurrence frequency of each sequence in the primary library).

Methods, reagents and apparatus for the synthesis (including high-throughput synthesis) of a nucleic acid are well known in the art, such as the phosphoramidite method and B3 Synthesizer from CustomArray. Methods, reagents and apparatus for the synthesis (including high-throughput synthesis) of a peptide are well known in the art, such as the carbodiimide method and SOPHAS of Zinsser Analytic. Methods, reagents and apparatus for the expression (including high-throughput expression) of a peptide are well known in the art. The expression system may be a cell expression system or a cell-free expression system (e.g., a ribosomal expression system). The cell can be a prokaryotic or a eukaryotic cell, and can be a bacterial, fungal, plant or animal (especially mammalian) cell.

In one embodiment, the predetermined length of a CDR amino acid sequence, the predetermined type of an allowable amino acid residue and the predetermined occurrence frequency of each amino acid residue at each position of the CDR amino acid sequence, the predetermined capacity of a primary CDR amino acid sequence library, and/or the predetermined capacity of a secondary CDR amino acid sequence library can be input based on an input file (e.g., an EXCEL file). In one embodiment, a CDR amino acid sequence library, a primary and/or secondary CDR amino acid sequence library, and/or a CDR nucleotide sequence library can be output based on an output file (e.g., an EXCEL file). In one embodiment, the output file is transmitted to a nucleic acid synthesis apparatus and/or a peptide expression apparatus to generate a corresponding nucleic acid and/or peptide library.

In this context, “about” means the error range well-recognized in the art, or ±10%, 5%, 3% or 1% of the indicated value.

### EXAMPLES

### Example 1

In the example, a (small, simple) antibody heavy chain CDR1 library was generated, with requirements as follows: the final library comprises 10000 amino acid sequences, the length of each sequence is 5 amino acid residues, and the allowable types of amino acids at each position and the ratio thereof are as shown in Table 1.

TABLE 1
Amino acid distribution set in heavy chain CDR1 library

| | H31 | H32 | H33 | H34 | H35 |
|---|---|---|---|---|---|
| Ala (A) | | | 45% | | |
| Asn (N) | | | | | 25% |
| Gly (G) | | | 55% | | |
| His (H) | | | | | 40% |
| Ile (I) | | | | 60% | |
| Met (M) | | | | 40% | |
| Ser (S) | 100% | | | | 35% |
| Tyr (Y) | | 100% | | | |

Step 1. All possible amino acid sequences were listed as an alternative sequence set. In this example, other than the amino acid distribution shown in Table 1, there are no additional limitations. The alternative sequence set consists of 12 sequences, as shown in Table 2.

TABLE 2
Alternative sequence set of
heavy chain CDR1 library

| No. | Sequence |
|---|---|
| 1 | SYAIN |
| 2 | SYAIH |
| 3 | SYAIS |
| 4 | SYAMN |
| 5 | SYAMH |
| 6 | SYAMS |
| 7 | SYGIN |
| 8 | SYGIH |
| 9 | SYGIS |
| 10 | SYGMN |
| 11 | SYGMH |
| 12 | SYGMS |

Step 2. For the library comprising 10000 sequences and having the amino acid distribution shown in Table 1, the given number of various amino acids at each position thereof was calculated, as shown in Table 3.

TABLE 3
Given number for each amino acid at each
position in heavy chain CDR1 library

| | H31 | H32 | H33 | H34 | H35 |
|---|---|---|---|---|---|
| Ala (A) | | | 4500 | | |
| Asn (N) | | | | | 2500 |
| Gly (G) | | | 5500 | | |
| His (H) | | | | | 4000 |
| Ile (I) | | | | 6000 | |
| Met (M) | | | | 4000 | |
| Ser (S) | 10000 | | | | 3500 |
| Tyr (Y) | | 10000 | | | |

Step 3. A sequence was randomly selected from an alternative sequence set for judging whether the addition of the sequence to a library will cause the number of certain amino acids at a certain position to exceed the given number of the amino acids at the position shown in Table 3, wherein if the number of certain amino acids at a certain position does not exceed the given number, then the sequence is added to the library, whereas if the number of certain amino acids at a certain position exceeds the given number, then the sequence is not added to the library and removed from the alternative sequence set.

The total number of sequences in the library was checked, wherein if the total number reached 10000, then the selection and storage tasks were completed, whereas if the total number did not reach 10000, then the selection and storage tasks were continued.

Step 4. After the above-mentioned operations, the actual number of various sequences in the generated library is as shown in Table 4. The library size of the example is 10000 sequences, and no expansion operation is required.

TABLE 4
Actual number for each sequence
in heavy chain CDR1 library

| No. | Sequence | Number |
|---|---|---|
| 1 | SYAIN | 610 |
| 2 | SYAIH | 943 |
| 3 | SYAIS | 962 |
| 4 | SYAMN | 607 |
| 5 | SYAMH | 708 |
| 6 | SYAMS | 670 |
| 7 | SYGIN | 661 |
| 8 | SYGIH | 1658 |
| 9 | SYGIS | 1166 |
| 10 | SYGMN | 622 |
| 11 | SYGMH | 691 |
| 12 | SYGMS | 702 |

By statistics, the distribution ratio of various amino acids at each position in the generated library is as shown in Table 5 and is exactly identical to the expected amino acid distribution in Table 1.

TABLE 5
Actual amino acid distribution in heavy chain CDR1 library

| | H31 | H32 | H33 | H34 | H35 |
|---|---|---|---|---|---|
| Ala (A) | | | 45.0% | | |
| Asn (N) | | | | | 25.0% |
| Gly (G) | | | 55.0% | | |
| His (H) | | | | | 40.0% |
| Ile (I) | | | | 60.0% | |
| Met (M) | | | | 40.0% | |
| Ser (S) | 100.0% | | | | 35.0% |
| Tyr (Y) | | 100.0% | | | |

Step 5. The amino acid sequences in the library were reverse-translated into DNA sequences.

Step 6. A high-throughput gene synthesis was performed with chips.

### Example 2

In the example, a (large, simple) antibody heavy chain CDR1 library was generated, with requirements as follows: the final library comprises 1 000 000 amino acid sequences, the length of each sequence is 5 amino acid residues, and the allowable types of amino acids at each position and the ratio thereof are as shown in Table 6. In this example, the sequence distribution in a primary library of 10000 sequences was determined and then expanded to a secondary library of 1,000,000 sequences.

TABLE 6
Amino acid distribution set in heavy chain CDR1 library

| | H31 | H32 | H33 | H34 | H35 |
|---|---|---|---|---|---|
| Ala (A) | | | 45% | | |
| Asn (N) | | | | | 25% |
| Gly (G) | | | 55% | | |
| His (H) | | | | | 40% |
| Ile (I) | | | | 60% | |
| Met (M) | | | | 40% | |
| Ser (S) | 100% | | | | 35% |
| Tyr (Y) | | 100% | | | |

Step 1. All possible amino acid sequences were listed as an alternative sequence set. In this example, other than the amino acid distribution shown in Table 6, there are no additional limitations. The alternative sequence set consists of 12 sequences, as shown in Table 7.

TABLE 7
Alternative sequence set of
heavy chain CDR1 library

| No. | Sequence |
|---|---|
| 1 | SYAIN |
| 2 | SYAIH |
| 3 | SYAIS |
| 4 | SYAMN |
| 5 | SYAMH |
| 6 | SYAMS |
| 7 | SYGIN |
| 8 | SYGIH |
| 9 | SYGIS |
| 10 | SYGMN |
| 11 | SYGMH |
| 12 | SYGMS |

Step 2. For the primary library comprising 10000 sequences and having the amino acid distribution shown in Table 6, the given number of various amino acids at each position thereof was calculated, and the results are as shown in Table 8.

TABLE 8
Given number for each amino acid at
each position in primary library

| | H31 | H32 | H33 | H34 | H35 |
|---|---|---|---|---|---|
| Ala (A) | | | 4500 | | |
| Asn (N) | | | | | 2500 |
| Gly (G) | | | 5500 | | |
| His (H) | | | | | 4000 |
| Ile (I) | | | | 6000 | |
| Met (M) | | | | 4000 | |
| Ser (S) | 10000 | | | | 3500 |
| Tyr (Y) | | 10000 | | | |

Step 3. A sequence was randomly selected from an alternative sequence set for judging whether the addition of the sequence to a primary library will cause the number of certain amino acids at a certain position to exceed the given number of the amino acids at the position shown in Table 8, wherein if the number of certain amino acids at a certain position does not exceed the given number, then the sequence is added to the primary library, whereas if the number of certain amino acids at a certain position exceeds the given number, then the sequence is not added to the primary library and removed from the alternative sequence set.

The total number of sequences in the primary library was checked, wherein if the total number reached 10000, then the selection and storage tasks were completed, whereas if the total number did not reach 10000, then the selection and storage tasks were continued.

Step 4. After the above-mentioned operations, the actual number and proportion of various sequences in the generated primary library are as shown in Table 9. The library size of the example is 1000000 sequences, and expansion operation is required. The actual proportion of various sequences in the primary library was used as the sampling probability of the secondary library.

Table 9: Actual number and proportion of each sequence in primary library

The proportion shown in Table 9 was used as the probability distribution, and 1000000 sequences were re-selected from the alternative sequence set to generate a secondary library. The actual number of various sequences in the secondary generated library is as shown in Table 10.

TABLE 10
Actual number for each sequence in
heavy chain CDR1 library

| No. | Sequence | Number |
|---|---|---|
| 1 | SYAIN | 61407 |
| 2 | SYAIH | 94304 |
| 3 | SYAIS | 96356 |
| 4 | SYAMN | 60931 |
| 5 | SYAMH | 70800 |
| 6 | SYAMS | 67183 |
| 7 | SYGIN | 65934 |
| 8 | SYGIH | 164791 |
| 9 | SYGIS | 116449 |
| 10 | SYGMN | 62122 |
| 11 | SYGMH | 68925 |
| 12 | SYGMS | 70798 |

By statistics, the distribution ratio of various amino acids at each position in the generated secondary library is as shown in Table 11 and is basically identical to the expected amino acid distribution in Table 6.

TABLE 11
Actual amino acid distribution in heavy chain CDR1 library

| | H31 | H32 | H33 | H34 | H35 |
|---|---|---|---|---|---|
| Ala (A) | | | 45.10% | | |
| Asn (N) | | | | | 25.04% |
| Gly (G) | | | 54.90% | | |
| His (H) | | | | | 39.88% |
| Ile (I) | | | | 59.92% | |
| Met (M) | | | | 40.08% | |
| Ser (S) | 100.00% | | | | 35.08% |
| Tyr (Y) | | 100.00% | | | |

Step 5. The amino acid sequences in the secondary library were reverse-translated into DNA sequences.

Step 6. A high-throughput gene synthesis was performed with chips.

The square of the coefficient of determination, i.e., R² was used to calculate the degree of agreement between the actual amino acid distribution of multiple selection positions H33, H34 and H35 and the expected amino acid distribution thereof. The calculated R² values for the positions are respectively: 0.9996 for H33; 0.9999 for H34; and 0.9998 for H35.

### Example 3

In the example, a (small, complex) antibody heavy chain CDR1 library was generated, with requirements as follows: the final library comprises 10000 amino acid sequences, the length of each sequence is 5 amino acid residues, and the allowable types of amino acids at each position and the ratio thereof are as shown in Table 12.

TABLE 12
Amino acid distribution set in heavy chain CDR1 library

| | H31 | H32 | H33 | H34 | H35 |
|---|---|---|---|---|---|
| Ala (A) | | 5.5% | 45.0% | | |
| Asn (N) | | 6.5% | | | 25.0% |
| Asp (D) | | 7.5% | | | |
| Gly (G) | | 8.5% | 55.0% | | |
| His (H) | | 9.5% | | | 40.0% |
| Ile (I) | | 10.5% | | 60.0% | |
| Leu (L) | | 11.5% | | | |
| Met (M) | | 12.5% | | 40.0% | |
| Ser (S) | 100.0% | 13.5% | | | 35.0% |
| Tyr (Y) | | 14.5% | | | |

Step 1. All the possible amino acid sequences were listed as alternative sequences. In this example, other than the amino acid distribution shown in Table 12, there are no additional limitations. The alternative sequence set consists of 120 sequences, as shown in Table 13.

TABLE 13
Alternative sequence set of
heavy chain CDR1 library

| No. | Sequence |
|---|---|
| 1 | SAAIS |
| 2 | SAAIN |
| 3 | SAAIH |
| 4 | SAAMS |
| 5 | SAAMN |
| 6 | SAAMH |
| 7 | SAGIS |
| 8 | SAGIN |
| 9 | SAGIH |
| 10 | SAGMS |
| 11 | SAGMN |
| 12 | SAGMH |
| 13 | SNAIS |
| 14 | SNAIN |
| 15 | SNAIH |
| 16 | SNAMS |
| 17 | SNAMN |
| 18 | SNAMH |
| 19 | SNGIS |
| 20 | SNGIN |
| 21 | SNGIH |
| 22 | SNGMS |
| 23 | SNGMN |
| 24 | SNGMH |
| 25 | SDAIS |
| 26 | SDAIN |
| 27 | SDAIH |
| 28 | SDAMS |
| 29 | SDAMN |
| 30 | SDAMH |
| 31 | SDGIS |
| 32 | SDGIN |
| 33 | SDGIH |
| 34 | SDGMS |
| 35 | SDGMN |
| 36 | SDGMH |
| 37 | SGAIS |
| 38 | SGAIN |
| 39 | SGAIH |
| 40 | SGAMS |
| 41 | SGAMN |
| 42 | SGAMH |
| 43 | SGGIS |
| 44 | SGGIN |
| 45 | SGGIH |
| 46 | SGGMS |
| 47 | SGGMN |
| 48 | SGGMH |
| 49 | SHAIS |
| 50 | SHAIN |
| 51 | SHAIH |
| 52 | SHAMS |
| 53 | SHAMN |
| 54 | SHAMH |
| 55 | SHGIS |
| 56 | SHGIN |
| 57 | SHGIH |
| 58 | SHGMS |
| 59 | SHGMN |
| 60 | SHGMH |
| 61 | SIAIS |
| 62 | SIAIN |
| 63 | SIAIH |
| 64 | SIAMS |
| 65 | SIAMN |
| 66 | SIAMH |
| 67 | SIGIS |
| 68 | SIGIN |
| 69 | SIGIH |
| 70 | SIGMS |
| 71 | SIGMN |
| 72 | SIGMH |
| 73 | SLAIS |
| 74 | SLAIN |
| 75 | SLAIH |
| 76 | SLAMS |
| 77 | SLAMN |
| 78 | SLAMH |
| 79 | SLGIS |
| 80 | SLGIN |
| 81 | SLGIH |
| 82 | SLGMS |
| 83 | SLGMN |
| 84 | SLGMH |
| 85 | SMAIS |
| 86 | SMAIN |
| 87 | SMAIH |
| 88 | SMAMS |
| 89 | SMAMN |
| 90 | SMAMH |
| 91 | SMGIS |
| 92 | SMGIN |
| 93 | SMGIH |
| 94 | SMGMS |
| 95 | SMGMN |
| 96 | SMGMH |
| 97 | SSAIS |
| 98 | SSAIN |
| 99 | SSAIH |
| 100 | SSAMS |
| 101 | SSAMN |
| 102 | SSAMH |
| 103 | SSGIS |
| 104 | SSGIN |
| 105 | SSGIH |
| 106 | SSGMS |
| 107 | SSGMN |
| 108 | SSGMH |
| 109 | SYAIS |
| 110 | SYAIN |
| 111 | SYAIH |
| 112 | SYAMS |
| 113 | SYAMN |
| 114 | SYAMH |
| 115 | SYGIS |
| 116 | SYGIN |
| 117 | SYGIH |
| 118 | SYGMS |
| 119 | SYGMN |
| 120 | SYGMH |

Step 2. For the library comprising 10000 sequences and having the amino acid distribution shown in Table 13, the given number of various amino acids at each position thereof was calculated, and the results are as shown in Table 14.

TABLE 14
Given number for each amino acid at each
position in heavy chain CDR1 library

| | H31 | H32 | H33 | H34 | H35 |
|---|---|---|---|---|---|
| Ala (A) | | 550 | 4500 | | |
| Asn (N) | | 650 | | | 2500 |
| Asp (D) | | 750 | | | |
| Gly (G) | | 850 | 5500 | | |
| His (H) | | 950 | | | 4000 |
| Ile (I) | | 1050 | | 6000 | |
| Leu (L) | | 1150 | | | |
| Met (M) | | 1250 | | 4000 | |
| Ser (S) | 10000 | 1350 | | | 3500 |
| Tyr (Y) | | 1450 | | | |

Step 3. A sequence was randomly selected from an alternative sequence set for judging whether the addition of the sequence to a library will cause the number of certain amino acids at a certain position to exceed the given number of the amino acids at the position shown in Table 14, wherein if the number of certain amino acids at a certain position does not exceed the given number, then the sequence is added to the library, whereas if the number of certain amino acids at a certain position exceeds the given number, then the sequence is not added to the library and removed from the alternative sequence set.

The total number of sequences in the library was checked, wherein if the total number reached 10000, then the selection and storage tasks were completed, whereas if the total number did not reach 10000, then the selection and storage tasks were continued.

Step 4. After the above-mentioned operations, the actual number of various sequences in the generated library is shown in Table 15. The library size of the example is 10000 sequences, and no expansion operation is required.

TABLE 15
Actual number for each sequence
in heavy chain CDR1 library

| No. | Sequence | Number |
|---|---|---|
| 1 | SAAIS | 38 |
| 2 | SAAIN | 45 |
| 3 | SAAIH | 45 |
| 4 | SAAMS | 54 |
| 5 | SAAMN | 65 |
| 6 | SAAMH | 45 |
| 7 | SAGIS | 41 |
| 8 | SAGIN | 52 |
| 9 | SAGIH | 49 |
| 10 | SAGMS | 35 |
| 11 | SAGMN | 43 |
| 12 | SAGMH | 38 |
| 13 | SNAIS | 48 |
| 14 | SNAIN | 49 |
| 15 | SNAIH | 70 |
| 16 | SNAMS | 57 |
| 17 | SNAMN | 53 |
| 18 | SNAMH | 48 |
| 19 | SNGIS | 56 |
| 20 | SNGIN | 42 |
| 21 | SNGIH | 59 |
| 22 | SNGMS | 63 |
| 23 | SNGMN | 56 |
| 24 | SNGMH | 49 |
| 25 | SDAIS | 61 |
| 26 | SDAIN | 61 |
| 27 | SDAIH | 70 |
| 28 | SDAMS | 72 |
| 29 | SDAMN | 58 |
| 30 | SDAMH | 60 |
| 31 | SDGIS | 66 |
| 32 | SDGIN | 61 |
| 33 | SDGIH | 72 |
| 34 | SDGMS | 58 |
| 35 | SDGMN | 56 |
| 36 | SDGMH | 55 |
| 37 | SGAIS | 90 |
| 38 | SGAIN | 74 |
| 39 | SGAIH | 76 |
| 40 | SGAMS | 72 |
| 41 | SGAMN | 86 |
| 42 | SGAMH | 77 |
| 43 | SGGIS | 69 |
| 44 | SGGIN | 58 |
| 45 | SGGIH | 61 |
| 46 | SGGMS | 70 |
| 47 | SGGMN | 51 |
| 48 | SGGMH | 67 |
| 49 | SHAIS | 102 |
| 50 | SHAIN | 70 |
| 51 | SHAIH | 92 |
| 52 | SHAMS | 97 |
| 53 | SHAMN | 64 |
| 54 | SHAMH | 81 |
| 55 | SHGIS | 90 |
| 56 | SHGIN | 63 |
| 57 | SHGIH | 79 |
| 58 | SHGMS | 76 |
| 59 | SHGMN | 58 |
| 60 | SHGMH | 78 |
| 61 | SIAIS | 100 |
| 62 | SIAIN | 75 |
| 63 | SIAIH | 91 |
| 64 | SIAMS | 87 |
| 65 | SIAMN | 65 |
| 66 | SIAMH | 73 |
| 67 | SIGIS | 141 |
| 68 | SIGIN | 67 |
| 69 | SIGIH | 155 |
| 70 | SIGMS | 73 |
| 71 | SIGMN | 54 |
| 72 | SIGMH | 69 |
| 73 | SLAIS | 107 |
| 74 | SLAIN | 70 |
| 75 | SLAIH | 127 |
| 76 | SLAMS | 56 |
| 77 | SLAMN | 59 |
| 78 | SLAMH | 83 |
| 79 | SLGIS | 186 |
| 80 | SLGIN | 61 |
| 81 | SLGIH | 164 |
| 82 | SLGMS | 76 |
| 83 | SLGMN | 65 |
| 84 | SLGMH | 96 |
| 85 | SMAIS | 128 |
| 86 | SMAIN | 54 |
| 87 | SMAIH | 104 |
| 88 | SMAMS | 88 |
| 89 | SMAMN | 70 |
| 90 | SMAMH | 62 |
| 91 | SMGIS | 205 |
| 92 | SMGIN | 56 |
| 93 | SMGIH | 277 |
| 94 | SMGMS | 64 |
| 95 | SMGMN | 67 |
| 96 | SMGMH | 75 |
| 97 | SSAIS | 115 |
| 98 | SSAIN | 80 |
| 99 | SSAIH | 105 |
| 100 | SSAMS | 77 |
| 101 | SSAMN | 69 |
| 102 | SSAMH | 71 |
| 103 | SSGIS | 173 |
| 104 | SSGIN | 76 |
| 105 | SSGIH | 374 |
| 106 | SSGMS | 81 |
| 107 | SSGMN | 71 |
| 108 | SSGMH | 58 |
| 109 | SYAIS | 103 |
| 110 | SYAIN | 72 |
| 111 | SYAIH | 113 |
| 112 | SYAMS | 72 |
| 113 | SYAMN | 77 |
| 114 | SYAMH | 67 |
| 115 | SYGIS | 180 |
| 116 | SYGIN | 51 |
| 117 | SYGIH | 481 |
| 118 | SYGMS | 73 |
| 119 | SYGMN | 76 |
| 120 | SYGMH | 84 |

By statistics, the distribution ratio of various amino acids at each position in the generated library is as shown in Table 16 and is almost identical to the expected amino acid distribution in Table 12.

TABLE 16
Actual amino acid distribution in heavy chain CDR1 library

| | H31 | H32 | H33 | H34 | H35 |
|---|---|---|---|---|---|
| Ala (A) | | 5.50% | 45.00% | | |
| Asn (N) | | 6.50% | | | 25.00% |
| Asp (D) | | 7.50% | | | |
| Gly (G) | | 8.51% | 55.00% | | |
| His (H) | | 9.50% | | | 40.00% |
| Ile (I) | | 10.50% | | 60.00% | |
| Leu (L) | | 11.50% | | | |
| Met (M) | | 12.50% | | 40.00% | |
| Ser (S) | 100.00% | 13.50% | | | 35.00% |
| Tyr (Y) | | 14.49% | | | |

The above results demonstrate that in the case that there are many optional sequences and the amino acid distribution is relatively complex, the method of the present invention can also obtain good results.

Step 5. The amino acid sequences in the library were reverse-translated into DNA sequences.

Step 6. A high-throughput gene synthesis was performed with chips.

The square of the coefficient of determination, i.e., R², was used to calculate the degree of agreement between the actual amino acid distribution of multiple selection positions H32, H33, H34 and H35 and the expected amino acid distribution thereof. The calculated R² values for the positions are respectively: 0.999998 for H32; 1.000000 for H33; 1.000000 for H34; and 1.000000 for H35.

1. A method for generating a primary CDR amino acid sequence library, comprising the steps of:
(1) determining all allowable CDR amino acid sequences according to a predetermined type of an allowable amino acid residue at each position of a CDR amino acid sequence, and according to a specified excluded sequence, to produce a set of alternative CDR amino acid sequences;
(2) determining an allowable number for each amino acid residue at each position of the CDR amino acid sequence in the primary CDR amino acid sequence library according to a predetermined capacity of the primary CDR amino acid sequence library and a predetermined occurrence frequency of each allowable amino acid residue at each position of the CDR amino acid sequence; and
(3) randomly selecting a CDR amino acid sequence from the set of alternative CDR amino acid sequences and adding the selected CDR amino acid sequence to the primary CDR amino acid sequence library and cycling same, until the cumulative number of CDR amino acid sequences added to the primary CDR amino acid sequence library reaches the predetermined capacity of the primary CDR amino acid sequence library, thereby generating the primary CDR amino acid sequence library, wherein
(3.1) when the set of alternative CDR amino acid sequences has not been emptied, a CDR amino acid sequence is randomly selected from the set of alternative CDR amino acid sequences, wherein
(3.1.1) when adding the selected CDR amino acid sequence to the primary CDR amino acid sequence library does not result in that the cumulative number of corresponding amino acid residues at any position exceeds the allowable number of the corresponding amino acid residues at the position, adding the selected CDR amino acid sequence to the primary CDR amino acid sequence library, or
(3.1.2) when adding the selected CDR amino acid sequence to the primary CDR amino acid sequence library results in that the cumulative number of corresponding amino acid residues at least one position exceeds the allowable number of the corresponding amino acid residues at the position, removing the selected CDR amino acid sequence from the set of alternative CDR amino acid sequences, or
(3.2) when the set of alternative CDR amino acid sequences has been emptied, a CDR amino acid sequence is randomly selected from the initial set of alternative CDR amino acid sequences.

2. The method of claim 1, further comprising generating a CDR nucleotide sequence library from the primary CDR amino acid sequence library by:
reverse-translating all the CDR amino acid sequences in the primary CDR amino acid sequence library into CDR nucleotide sequences, thereby generating the CDR nucleotide sequence library.

3. (canceled)

4. The method of claim 2, further comprising generating a CDR peptide library from the CDR nucleotide sequence library by:
(5) synthesizing CDR nucleic acids according to all the CDR nucleotide sequences in the CDR nucleotide sequence library, thereby generating a CDR nucleic acid library; and
(6) expressing all the CDR nucleic acids in the CDR nucleic acid library in an expression system, thereby generating the CDR peptide library.

5. The method of claim 1, further comprising generating a CDR peptide library from the CDR amino acid sequence library by:
(4) synthesizing CDR peptides according to all the CDR amino acid sequences in the CDR amino acid sequence library, thereby generating the CDR peptide library.

6. (canceled)

7. The method of claim 1, further comprising generating a large CDR amino acid sequence library from a primary CDR amino acid sequence library, comprising the steps of:
(4) determining an occurrence frequency of each CDR amino acid sequence in the primary CDR amino acid sequence library; and
(5) according to the occurrence frequency of each CDR amino acid sequence in the primary CDR amino acid sequence library, randomly selecting a sequence from the initial set of alternative CDR amino acid sequences and adding the selected sequence to a secondary CDR amino acid sequence library, until the cumulative number of CDR amino acid sequences added to the secondary CDR amino acid sequence library reaches the predetermined capacity of the secondary CDR amino acid sequence library, thereby generating the secondary CDR amino acid sequence library, and thereby generating the large CDR amino acid sequence library.

8-11. (canceled)

12. The method of claim 1, wherein the predetermined capacity of the primary CDR amino acid sequence library is 1,000 to 10,000 amino acid sequences.

13. The method of claim 7, wherein the predetermined capacity of the secondary CDR amino acid sequence library is 10 to 1000 times the predetermined capacity of the primary CDR amino acid sequence library.

14. The method of claim 1, wherein the length of the CDR amino acid sequence is 3 to 10 amino acid residues, and wherein the CDR amino acid sequence comprises 3 to 10 variable positions.

15. (canceled)

16. The method of claim 1, wherein the initial set of alternative CDR amino acid sequences comprises 10 to 1000 allowable CDR amino acid sequences.

17. (canceled)

18. A device for generating a CDR nucleotide sequence library comprising the following apparatuses:
an input apparatus, which is configured for inputting a predetermined length of a CDR amino acid sequence, a predetermined type of an allowable amino acid residue and a predetermined occurrence frequency of each amino acid residue at each position of the CDR amino acid sequence, and/or a predetermined capacity of the CDR amino acid sequence library;
a processing apparatus, which is configured to be used for performing the operations of:
(1) determining all allowable CDR amino acid sequences according to a predetermined type of an allowable amino acid residue at each position of a CDR amino acid sequence, and optionally according to a specified excluded sequence, to produce a set of alternative CDR amino acid sequences;
(2) determining an allowable number for each amino acid residue at each position of the CDR amino acid sequence in a primary CDR amino acid sequence library according to a predetermined capacity of the primary CDR amino acid sequence library and a predetermined occurrence frequency of each allowable amino acid residue at each position of the CDR amino acid sequence;
(3) randomly selecting a CDR amino acid sequence from the set of alternative CDR amino acid sequences and adding the selected CDR amino acid sequence to the primary CDR amino acid sequence library and cycling same, until the cumulative number of CDR amino acid sequences added to the primary CDR amino acid sequence library reaches the predetermined capacity of the primary CDR amino acid sequence library, thereby generating the primary CDR amino acid sequence library, wherein
(3.1) when the set of alternative CDR amino acid sequences has not been emptied, a CDR amino acid sequence is randomly selected from the set of alternative CDR amino acid sequences, wherein
(3.1.1) when adding the selected CDR amino acid sequence to the primary CDR amino acid sequence library does not result in that the cumulative number of corresponding amino acid residues at any position exceeds the allowable number of the corresponding amino acid residues at the position, adding the selected CDR amino acid sequence to the primary CDR amino acid sequence library, or
(3.1.2) when adding the selected CDR amino acid sequence to the primary CDR amino acid sequence library results in that the cumulative number of corresponding amino acid residues at least one position exceeds the allowable number of the corresponding amino acid residues at the position, removing the selected CDR amino acid sequence from the set of alternative CDR amino acid sequences, or
(3.2) when the set of alternative CDR amino acid sequences has been emptied, a CDR amino acid sequence is randomly selected from the initial set of alternative CDR amino acid sequences; and
(4) reverse-translate all the CDR amino acid sequences in the primary CDR amino acid sequence library into CDR nucleotide sequences, thereby generating the CDR nucleotide sequence library; and
an output apparatus, which is configured for outputting the CDR nucleotide sequence library.

19. (canceled)

20. The device of claim 18, wherein the device is further configured for generating a CDR peptide library from the CDR nucleotide sequence library, wherein the device further comprising the following apparatus:
a nucleic acid synthesis apparatus, which is configured for synthesizing CDR nucleic acids according to all the CDR nucleotide sequences in the CDR nucleotide sequence library, thereby generating a CDR nucleic acid library; and
a peptide expression apparatus, which is configured for expressing all the CDR nucleic acids in the CDR nucleic acid library in an expression system, thereby generating the CDR peptide library.

21. The device of claim 18, wherein the device is further configured for generating a CDR peptide library from the primary CDR amino acid sequence library, wherein the device further comprises the following apparatus:
a peptide synthesis apparatus, which is configured for synthesizing CDR peptides according to all the CDR amino acid sequences in the primary CDR amino acid sequence library, thereby generating the CDR peptide library.

22. (canceled)

23. The device of claim 18, wherein the device is further configured for generating a large CDR amino acid sequence library from the primary CDR amino acid sequence library by:
an input apparatus, which is configured for inputting a predetermined length of a CDR amino acid sequence, a predetermined type of an allowable amino acid residue and a predetermined occurrence frequency of each amino acid residue at each position of the CDR amino acid sequence, a predetermined capacity of the primary CDR amino acid sequence library, and/or a predetermined capacity of a secondary CDR amino acid sequence library;
the processing apparatus, which is further configured to
(4) determine an occurrence frequency of each CDR amino acid sequence in the primary CDR amino acid sequence library; and
(5) according to the occurrence frequency of each CDR amino acid sequence in the primary CDR amino acid sequence library, randomly select a sequence from the initial set of alternative CDR amino acid sequences and add the selected sequence to a secondary CDR amino acid sequence library, until the cumulative number of CDR amino acid sequences added to the secondary CDR amino acid sequence library reaches the predetermined capacity of the secondary CDR amino acid sequence library, thereby generating the secondary CDR amino acid sequence library, and thereby generating the large CDR amino acid sequence library; and
the output apparatus, which is further configured for outputting the large CDR amino acid sequence library.

24-27. (canceled)

28. The device of claim 18, wherein the predetermined capacity of the primary CDR amino acid sequence library is 1,000 to 10,000 amino acid sequences.

29. The device of claim 23, wherein the predetermined capacity of the secondary CDR amino acid sequence library is 10 to 1000 times the predetermined capacity of the primary CDR amino acid sequence library.

30. The device of claim 18, wherein the length of the CDR amino acid sequence is 3 to 10 amino acid residues, and wherein the CDR amino acid sequence comprises 3 to 10 variable positions.

31. (canceled)

32. The device of claim 18, wherein the initial set of alternative CDR amino acid sequences comprises 10 to 1000 allowable CDR amino acid sequences.

33. The device of claim 18, further comprising a storage apparatus, which is configured to store an algorithm for performing the operations.

34. A computer program product, comprising a computer program instruction for operating the device of claim 18.

35. A storage apparatus for storing the computer program product of claim 34.
